(19) United States
(12) Patent Application Publication
(10) Pub. No.: US 2003/0054993 A1
(43) Pub. Date: Mar. 20, 2003

(54) Implantation serine proteinases

(76) Inventors: Derrick E. Rancourt, Calgary (CA); Susan L. Rancourt, Calgary (CA); Colleen M. O'Sullivan, Calgary (CA)

Correspondence Address: BURNS DOANE SWECKER & MATHIS L L P, POST OFFICE BOX 1404, ALEXANDRIA, VA, 22313-1404, US

(21) Appl. No.: 10/117,323

(22) Filed: Apr. 8, 2002

Related U.S. Application Data
(60) Provisional application No. 60/281,724, filed on Apr. 6, 2001.
(60) Provisional application No. 60/294,736, filed on May 30, 2001.
(60) Provisional application No. 60/350,962, filed on Jan. 25, 2002.

(51) Int. Cl. A61K 48/00; A61K 38/17; C07H 21/04; C12P 21/02; C12N 9/64; C12N 5/06

(52) U.S. Cl. 514/12; 514/44; 435/226; 435/69.1; 435/320.1; 435/325; 536/23.2

(57) ABSTRACT

This invention provides two novel serine proteinases that are important for female fertility, particularly in the process of hatching and implantation. These proteinases, as well as the nucleic acids, fragments, analogs, and/or inhibitors thereof, can be used to modulate hatching, implantation and female fertility in general.

### RELATED APPLICATION

This application claims the benefit of U.S. Provisional Application Serial Nos. 60/281,724, filed Apr. 6, 2001; 60/294,736, filed May 30, 2001; and 60/350,962, filed Jan. 25, 2002; all of which are hereby incorporated by reference in their entirety.

### FIELD OF THE INVENTION

This invention relates to proteinases that are involved in hatching and implantation of the embryo, and their use in contraception or to enhance fertility.

### REFERENCES

U.S. Pat. No. 5,023,252.

Afonso, S. et al. (1997). The expression and function of cystatino C and cathepsin B and cathepsin L during mouse embryo implantation and placentation. Development 124: 3415-3425.

Alexander, C. M. et al. (1996). Expression and function of matrix metalloproteinases and their inhibitors at the maternal-embryonic boundary during mouse embryo implantation. Development 122: 1723-1736.

Altschul, S. F. et al., (1997). Gapped BLAST and PSI-BLAST: a new generation of protein database search programs. Nucleic Acids Research 25: 3389-3402.

Arcellana-Panlilio, M. Y. and G. A. Schultz (1993). Analysis of messenger RNA Methods in Enzymology. 225: 303-328.

Baker, J. et al. (1993). Role of insulin-like growth factors in embryonic and postnatal growth. Cell 75: 73-82.

Behrendtsen, 0. et al. (1992). Metalloproteinases mediate extracellular matrix degradation by cells from mouse blastocyst outgrowths. Development 114: 447-456.

Behrendtsen, O. et al. (1995). Cooperative interactions between extracellular matrix, integrins and parathyroid hormone-related peptide regulate parietal endoderm differentiation in mouse embryos. Development 121: 4137-4148.

Bider, D. et al. (1997). Assisted hatching by zona drilling of human embryos in women of advanced age. Human Reproduction 12: 317-20.

Burge, C and Karlin, S. (1997). Prediction of complete gene structures in human genomic DNA. J. Mol. Biol. 268: 78-94.

Carmeliet, P. et al. (1994). Physiological consequences of loss of plasminogen activator gene function in mice. Nature 368: 419-424.

Carson, D. D. et al. (1993). Heparin sulfate proteoglycan (perlecan) expression by mouse embryos during acquisition of attachment competence. Developmental Biology 155: 97-106.

Carson, D. D. et al. (2000). Embryo implantation. Developmental Biology 223: 217-37.

Compton, S. J. et al. (1998). The role of mast cell tryptase in regulating endothelial cell proliferation, cytokine release, and adhesion molecule expression: tryptase induces expression of mRNA for IL-1 beta and IL-8 and stimulates the selective release of IL-8 from human umbilical vein endothelial cells. Journal of Immunology 161: 1939-1946.

Daniels, R J, Peden, J F, Lloyd, C, Horsley, S W, Clark, K, Tufarelli, C, Kearney L, Buckle, V J, Doggett, N A, Flint, J and Higgs DR. (2001). Sequence, structure and pathology of the fully annotated terminal 2 Mb of the short arm of human chromosome 16. Hum. Mol. Genet. 10: 339-352.

Danielsson, K. G. et al. (1997). The effect of various doses of mifepristone on endometrial leukaemia inhibitory factor expression in the midluteal phase—an immunohistochemical study. Human Reproduction 12: 1293-1297.

Das, S. K. et al. (1994). Heparin-binding EGF-like growth factor gene is induced in the mouse uterus temporally by the blastocyst solely at the site of its apposition: a possible ligand for interaction of blastocyst EGF receptor in implantation. Development 120: 1071-1083.

De Vos, A. and A. Van Steirteghem (2000). Zona hardening, zona drilling and assisted hatching: new achievements in assisted reproduction. Cells Tissues and Organs 166: 220-227.

Denker , H. W. (1977). Implantation: the role of proteinases and blockage of implantation by proteinase inhibitors. Advances in Anatomy, Embryology and Cell Biology 53: 3-123.

Duc-Goiran, P. et al. (1999). Embryo-maternal interactions at the implantation site: a delicate equilibrium. European Journal of Obstetrics, Gynecological and Reproductive Biology 83: 85-100.

Erbach, G. T. et al. (1994). Differential growth of the mouse preimplantation embryo in chemically defined media. Biology of Reproduction 50: 1027-33.

Farach, M. C. et al. (1987). Heparin/heparan sulfate is involved in attachment and spreading of mouse embryos in vitro. Developmental Biology 123: 407-410.

Finn, C. A. (1966). Endocrine control of endometrial sensitivity during the induction of the decidual cell reaction in the mouse. Journal of Endocrinology 36: 239-248.

Fong, C.-Y. et al. (1998). Blastocyst transfer after enzymatic treatment of the zona pellucida: improving in-vitro fertilization and understanding implantation. Human Reproduction 13 2926-2932.

Ghosh, D. et al. (1998). Effect of early luteal phase administration of mifepristone (RU486) on leukaemia inhibitory factor, transforming growth factor beta and vascular endothelial growth factor in the implantation stage endometrium of the rhesus monkey. Journal of Endocrinology 157: 115-25.

Gonzales, D. S. and B. D. Bavinster (1995). Zona pellucida escape by hampster blastocysts in vitro is delayed and morphologically different compared to zona escape in vivo. Biology of Reproduction 52: 470-480.

Gray, A. et al. (2000). Ovine uterine gland knock-out model: effects of gland ablation on the estrous cycle. Biology of Reproduction 62: 448-456.

Greb, R. R. et al. (1999). Disparate actions of mifepristone (RU486) on glands and stroma in the primate endometrium. Human Reproduction 14: 198-206.

Harlow, E. et al. (1988). “Antibodies, A Laboratory Manual”, Cold Spring Harbor Laboratory, NY.

Harvey, M. B. et al. (1995). Proteinase expression in early mouse embryos is regulated by leukaemia inhibitory factor and epidermal growth factor. Development 121: 1005-1014.

Higgins, D. G. (1994). CLUSTAL V: multiple alignment of DNA and protein sequences. Methods in Molecular Biology 25: 307-318. Hogan, B. et al., (1994). “Manipulating the Mouse Embryo”, A Laboratory Manual, Cold Spring Harbor Laboratory Press, Cold Spring Harbor, New York.

Huang, C. et al. (2000). Formation of enzymatically active, homotypic, and heterotypic tetramers of mouse mast cell tryptases. Dependence on a conserved Trp-rich domain on the surface. Journal of Biological Chemistry 275: 351-358.

Huet-Hudson, Y. M. et al. (1989). Cell type-specific localization of c-Myc protein in the mouse uterus: modulation by steroid hormones and analysis of the peri-implantation period. Endocrinology 125: 1683-90.

Hwang, S. et al. (2000). Intactness of zona pellucida does not affect the secretion of a trypsin-like protease from mouse blastocyst. Journal of Korean Medical Science 15: 529-532.

Jeziorska, M. et al. (1996). Immunolocalization of the matrix metalloproteases gelatinase B and stromelysin 1 in human endometrium throughout the menstrual cycle. Journal of Reproduction and Fertility 107: 43-51.

Jones, D. H. et al. (1997). Embryonic expression of the putative gamma subunit of the sodium pump is required for acquisition of fluid transport capacity during mouse blastocyst development. Journal of Cell Biology 139: 1545-1552.

Joshi, M. S. and I. M. Murray (1974). Immunological studies of the rat uterine fluid peptidase. Journal of Reproduction and Fertility 37: 361-365.

Keski-Oja, J. et al. (1992). Proteolytic processing of the 72,000-Da type IV collagenase by urokinase plasminogen activator. Experimental Cell Research 202: 471-476.

Kirby, D. R. et al. (1967). On the orientation of the implanting blastocyst. Journal of Embryology and Experimental Morphology 17: 527-532.

Kubo, H. et al. (1981). Inhibition of mouse blastocyst attachment and outgrowth by protease inhibitors. Journal of Experimental Zoology 216: 445-451.

Lefebvre, O. et al. (1995). Developmental expression of mouse stromelysin 3 mRNA. Development 121: 947-955.

Lindstedt, K. A. et al. (1998). Regulation of the activity of secreted human lung mast cell tryptase by mast cell proteoglycans. Biochemica et Biophysica Acta 1425: 617-627.

Liu, C. Q. et al. (1999). Mifepristone regulation of leukemia inhibitory factor and uterine receptivity in rabbits. Contraception 60: 309-312.

Lohi, J. et al. (1992). Pericellular substrates of human mast cell tryptase: 72,000 dalton gelatinase and fibronectin. Journal of Cell Biology 50: 337-349.

Lutzelschwab, C. et al. (1997). Secretory granule proteases in rat mast cells. Cloning of 10 different serine proteases and carboxypeptidase A from various rat mast cell populations. Journal of Experimental Medicine 185: 13-29.

Martin, L. et al. (1973). The inhibition of progesterone of uterine epithelial proliferation in the mouse. Journal of Endocrinology 57: 549-554.

Milligan, S. R. et al. (1995). The minimum requirements for oestradiol to induce uterine sensitivity for implantation and decidualization in mice. Human Reproduction 10: 1502-1506.

Mintz, B. (1972). Implantation initiating factor from mouse uterus. In Biology of Mammalian Fertilization and Implantation. pp. 343-356 Eds. K. S. Moghissi and E. S. Hafz. Thomas Springfield, Ill.

Mirza, H. et al. (2000). Mitogenic responses through the proteinase activated receptor-2 are induced by expressed forms of mast cell alpha- or beta-tryptases. Blood 90: 3914-22.

Montag, M. et al. (2000). Significance of the number of embryonic cells and the state of the zona pellucida for hatching in mouse blastocysts in vitro versus in vivo. Biology of Reproduction 62: 1738-1744.

Muller-Newen, G. et al. (1996). Soluble receptors for cytokines and growth factors. International Archives of Allergy and Immunology 111: 99-106.

Orsini, M. W. and A. McLaren (1967). Loss of the zona pellucida in mice, and the effect of tubal ligation and ovariectomy. Journal of Reproduction and Fertility 13: 485-499.

Pampfer, S. et al. (1991). Expression of colony stimulating factor 1 (CSF-1) messenger RNA in human endometrial glands during the menstrual cycle: molecular cloning of a novel transcript that predicts a cell surface form of CSF-1. Molecular Endocrinology 5: 1931-1938.

Paria, B. C. et al. (1993). Blastocyst's state of activity determines the “window” of implantation in the receptive mouse uterus. Proceeding of the National Academy of Science USA 90: 10159-10162.

Paria, B. C. et al. (1999). Heparin-binding EGF-like growth factor interacts with mouse blastocysts independently of ErbB1: a possible role for heparan sulfate proteoglycans and ErbB4 in blastocyst implantation. Development 126: 1997-2005.

Peitsch, M. C., Schwede, T. and Guex, N. (2000). Automated protein modelling—the proteome in 3D. Pharmacogenomics 1: 257-266.

Perona, R. M. and P. M. Wassarman (1986). Mouse blastocysts hatch in vitro by using a trypsin-like proteinase associated with cells of mural trophoectoderm. Developmental Biology 114: 42-52.

Pinsker, M. C. et al. (1974). Implantation associated proteinase in mouse uterine fluid. Developmental Biology 38: 285-290.

Pollard, J. W. et al. (1991). A pregnancy defect in the osterpetrotic (op/op) mouse demonstrates the requirements for CSF-1 in female fertility. Developmental Biology 148: 273-83.

Prendergast, J. A. et al. (1991). Structure and evolution of the cytotoxic cell proteinase genes CCP3, CCP4 and CCP5. Journal of Molecular Biology 220: 867-875.

Psychoyos, A. (1973). Endocrine control of egg implantation. In Handbook of Physiology pp. 187-215 Eds. R. O. Greep, E. G. Astwood, S. R. Geiger, American Physiological Society, Washington, DC.

Raab, G. et al. (1996). Mouse preimplantation blastocysts adhere to cells expressing the transmembrane form of heparin-binding EGF-like growth factor. Development 122: 637-645.

Rancourt, S. L. and D. E. Rancourt (1997). Murine subtilisin-like proteinase SPC6 is expressed during embryonic implantation, somitogenesis and skeletal formation. Developmental Genetics 21: 75-81. Rathjen, P. D. et al. (1990). Developmentally programmed induction of differentiation inhibiting activity and the control of stem cell populations. Genes and Development 4: 2308-2318.

Regenstreif, L. J. and J. Rossant (1989). Expression of the c-fins proto-oncogene and of the cytokine, CSF-1, during mouse embryogenesis. Developmental Biology 133: 284-294.

Remington's Pharmaceutical Sciences (19^{th} Ed., 1995), Mack Publishing Company, Philadelphia Pa.

Rinkenberger, J. L. et al. (1997). Molecular genetics of implantation in the mouse. Developmental Genetics 21: 6-20.

Rosenfeld, M. G. and M. S. Joshi (1981). Effect of a rat uterine fluid endopeptidase on lysis of the zona pellucida. Journal of Reproduction and Fertility 62: 199-203.

Rutanen, E. M. (2000). Insulin-like growth factors in obstetrics. Current Opinion in Obstetrics and Gynecology 12: 163-168.

Sambrook, J. et al. (1989). Molecular Cloning: A Laboratory Manual Cold Spring Harbor Laboratory Press, Cold Spring Harbor, New York.

Sanderson, P. E. (1999). Small, noncovalent serine protease inhibitors. Medical Research Reviews 19: 179-197.

Sawada, H. et al. (1990). Trypsin-like hatching protease from mouse embryos: evidence for the presence in culture media and it enzymatic properties. Journal of Experimental Zoology 254: 83-87.

Schlingensiepen, K. H. and W. Brysch (1992). Phosphorothioate oligomers: Inhibitors of oncogene expression in tumor cells as a tool for gene function analysis. In Gene Regulation: Biology of Antisense RNA and DNA pp. 317-328 Eds. R. Erickson and J. Izant. Raven Press, New York.

Singh et al. (1999). Advances in vaccine adjuvants. Nat. Biotechnol. 17(11):1075-81.

Smyth, M. J. et al. (1996). Granzymes: a variety of serine protease specificities encoded by genetically distinct subfamilies. Journal of Leukocyte Biology 60: 555-562.

Song, H. et al. (2000). Dysregulation of EGF family of growth factors and COX-2 in the uterus during the preattachment and attachment reactions of the blastocyst with the luminal epithelium correlates with implantation failure in LIF-deficient mice. Molecular Endocrinology 14: 1147-61.

Stewart, C. L. et al. (1992). Blastocyst implantation depends on maternal expression of leukemia inhibitory factor. Nature 359: 76-79.

Taipale, J. and J. Keski-Oja (1997). Growth factors in the extracellular matrix. FASEB Journal 11: 51-59.

Teesalu, T. et al. (1996). Embryo implantation in mouse: fetomaternal coordination in the pattern of expression of uPA, uPAR, PAI-1 and alpha 2MR/LRP genes. Mechanisms of Development 56: 103-116.

Vu, T.H. et al. (1997). Identification and cloning of the membrane-associated serine proteinase, hepsin, from mouse preimplantation embryos. Journal of Biological Chemistry 272: 31315-31320.

Vu, T. H. et al. (1998). MMP-9/gelatinase B is a key regulator of growth plate, angiogenesis and apoptosis of hypertrophic chondrocytes. Cell 93: 411-422.

Wang, J. et al. (2000). Acceleration of trophoblast differentiation by heparin-binding EGF-like growth factor is dependent on the stage-specific activation of calcium influx by ErbB receptors in developing mouse blastocysts. Development 127: 33-44.

Werb, Z. et al. (1992). Expression and function of matrix metalloproteases in development. Matrix s1: 337-343.

Woltjen, K., Bain, G. and Rancourt, D.E. (2000). Retro-recombination screening of a mouse embryonic stem cell genomic library. Nucl. Acids Res. 28E41:1-7.

Wu, Q. et al. (1998). Generation and Characterisation of mice deficient in hepsin, a hepatic transmembrane serine protease. Journal of Clinical Investigation 101: 321-326.

Yoshinaga, K. and C. E. Adams (1966). Delayed implantation in the spayed, progesterone-treated adult mouse. Journal of Reproduction and Fertility 12: 593-595.

Zhang, J. G. et al. (1998). Identification and characterization of two distinct forms of gp13. and a soluble form of leukemia inhibitory factor receptor a-chain in normal human urine and plasma. Journal of Biological Chemistry 273: 10798-10805.

All of the above publications, patents and patent applications are herein incorporated by reference in their entirety to the same extent as if the disclosure of each individual publication, patent application or patent was specifically and individually indicated to be incorporated by reference in its entirety.

### BACKGROUND OF THE INVENTION

Successful implantation and subsequent pregnancy require the co-ordination of endometrial and blastocystic factors, to enable the correct attachment of the embryo and its subsequent invasion into maternal deciduum (for review see Rinkenberger et al., 1997; Carson et al., 2000). As the fertilized egg approaches the uterus it undergoes numerous divisions to form a blastocyst. Simultaneously, the maternal deciduum proliferates and becomes receptive to the attachment of trophoblast cells in a brief period called the “implantation window” (Psychoyos, 1973; Paria et al., 1993).

In the mouse, the hormones estrogen and progesterone are necessary to synchronize the interaction of the embryo and uterus. Estrogen that was released prior to ovulation stimulates the differentiation of uterine lumenal and endometrial epithelia on the first two days of pregnancy (Martin et al., 1973). By day three, rising levels of progesterone prompt stromal cell proliferation. On day four a preimplantation surge of estrogen (Huet-Hudson et al., 1989) makes the uterus responsive to tactile stimuli, either naturally by an embryo or artificially by an oil drop (Finn, 1966). If this surge does not occur (i.e. in ovariectomized females), hatched blastocysts are unable to attach and lie dormant in the uterus (Paria et al., 1993). The block in implantation can be overcome, within twenty days, by administration of estrogen, but only if preceded by progesterone priming for 24-48 hours (Yoshinaga and Adams, 1966).

In response to global regulation of implantation by hormones, cytokines exhibit local autocrine/paracrine effects and create a dialogue that operates largely between the endometrial glands, the lumenal epithelium and the embryo. This dialogue is mediated via several cytokine networks including EGF, LIF, CSF and IGF (Das et al., 1994; Stewart et al., 1992; Pollard et al., 1991; Regenstreif et al., 1989; Baker et al., 1993). In the early stages of pregnancy, prior to the establishment of the placenta, the endometrial glands serve as an important signaling center producing key factors and receptors. In response to the estrogen spike, for example, LIF is secreted from the endometrial gland and into the uterine lumen where it interacts with LIF-ra to facilitate the expression of tethered EGF ligands on the surface of the luminal epithelium (Song et al., 2000). In turn, these EGF ligands mediate blastocyst apposition via their interaction with the EGF receptor, ErbB4, which lies on the trophectodermal surface (Paria et al., 1999; Wang et al., 2000). CSF is also secreted from the endometrial gland in response to the oestrogen spike, and signals the embryonic receptor c-fms to stimulate trophoblast invasion (Pollard et al., 1991). Before attaching to the deciduum the blastocyst must also shed its proteinaceous sheath, the zona pellucida (zona). Thinning of the zona precedes hatching and is thought to be the result of both internal pressure from the growth of the blastocyst and the presence of uterine and embryo-derived “lysins” (Montag et al., 2000). An embryo-derived extracellular “trypsin-like” activity, required for the completion of hatching in vitro, has been bistochemically localized to the abembryonic pole where hatching is initiated (Perona and Wassarman, 1986; Sawada et al., 1990; Hwang et al., 2000). This apical surface is the first to become adhesive in utero and orients the blastocyst within the implantation chamber (Kirby et al., 1967).

After release from the zona, several extracellular matrix proteins promote blastocyst attachment and outgrowth in vitro (Carson et al., 1993). Heparin sulphate proteoglycan for example, is localized on the surface of abembryonic trophoblasts. Attachment and outgrowth of blastocysts in vitro is inhibited by heparinase or soluble heparin (Farach et al., 1987). Localized heparin sulfate may also facilitate the embryo/uterine dialog and blastocystic implantation competence, through the localized secretion of maternal heparin binding-epidermal growth factor (HB-EGF). Secreted HB-EGF promotes blastocyst hatching and outgrowth in vitro (Das et al., 1994). A transmembrane form of HB-EGF expressed on the surface of uterine epithelia, may mediate blastocyst adherence through this localized heparin sulfate proteoglycan and apically expressed EGF receptor, ErbB4 (Raab et al., 1996; Paria et al., 1999; Wang et al., 2000).

The embryo-uterine interaction and the integration of the embryo into the maternal crypt are also mediated by extracellular matrix-degrading proteinase that are secreted by the invading trophoblasts. On day 5 of embryogenesis, blastocystic urokinase plasminogen activator (uPA) occupies receptors on the trophoblast cell surface, where it is thought to activate ubiquitous plasminogen and initiate decidual extracellular matrix (ECM) degradation (Teesalu et al., 1996). Plasmin is also thought to activate trophoblastic MMP9, a matrix metalloproteinase that cleaves several ECM components which is suggested to give the embryo its invasive character (Harvey et al., 1995; Alexander et al., 1996).

Although inhibitor studies suggest that both uPA and MMP9 are important for blastocyst outgrowth during implantation (Behrendtsen et al., 1992; Werb et al., 1992), targeted mutagenesis indicates that either proteinase is dispensable (Carmeliet et al., 1994; Vu et al., 1998). These latter observations suggest that other proteinases may be involved in implantation and can somehow substitute for the missing enzyme activity. Some of these additional proteinases have recently been identified (Lefebvre et al., 1995; Afonso et al., 1997; Vu et al., 1997), but their roles in implantation have not been confirmed. Proteinase players in the implantation process, as well as their therapeutic uses, remain to be identified.

### SUMMARY OF THE INVENTION

We identified two novel serine proteinases, Implantation Serine Proteinase (ISP) 1 and 2, which are expressed at the implantation site of embryo. Expression pattern and/or antisense analyses indicate that ISP1 and ISP2 are important for hatching and/or implantation of the embryo. Furthermore, immunization of female rnice with ISP1 and ISP2 resulted in a significant decrease in the number of embryos successfully implanted.

Accordingly, one aspect of the present invention provides an isolated nucleic acid encoding an Implantation Serine Proteinase (ISP) protein, which possesses a biological activity of ISP1 or ISP2, as well as a substantial sequence identity with the cDNA sequence encoding ISP1 or ISP2 (SEQ ID Nos: 1 or 2). The sequence identity with SEQ ID NO:1 or NO:2 is preferably at least about 60%, more preferably at least about 70%, yet more preferably at least about 80%, and most preferably at least about 90%. In particular, the isolated DNA comprises SEQ ID NO:1 or NO:2.

Also provided is an isolated nucleic acid that is capable of hybridizing to SEQ ID NO: 1, SEQ ID NO:2, or their complements, at a stringency equivalent to 0.5×SSC and 50° C. The hybridization stringency is preferably equivalent to 0.5×SSC and 55° C., more preferably equivalent to 0.0×SSC and 55° C., and most preferably equivalent to 0.1×SSC and 60° C.

Another aspect of the present invention provides a vector, preferably an expression vector, that comprises the nucleic acid as described above. Also provided are cells comprising such a vector. The cells may be prokaryotic or eukaryotic. Examples of host cells include bacterial, yeast, insect and mammalian cells.

Another aspect of the present invention provides a purified ISP protein, which protein possesses a biological activity of ISP1 or ISP2, as well as a substantial sequence identity with ISP1 (SEQ ID NO:3) or ISP2 (SEQ ID NO:4). In particular, the protein is a recombinant protein. The sequence identity with SEQ ID NO:3 or NO:4 is preferably at least about 60%, more preferably at least about 70%, yet more preferably at least about 80%, and most preferably at least about 90%. In particular, the protein comprises SEQ ID NO:3 or NO:4.

Another aspect of the present invention provides a method for producing a recombinant ISP protein, comprising constructing an expression vector comprising a DNA encoding an ISP protein, introducing the expression vector into a suitable cell and selecting transformants, culturing the transformants under conditions that result in production of the ISP protein, and recovering the ISP protein. The DNA sequence has a sequence identity with SEQ ID NO:1 or NO:2 of preferably at least about 60%, more preferably at least about 70%, yet more preferably at least about 80%, and most preferably at least about 90%. In particular, the DNA comprises SEQ ID NO:1 or NO:2. Alternatively, the DNA sequence is capable of hybridizing to SEQ ID NO:1 or SEQ ID NO:2 at a stringency equivalent to 0.5×SSC and 50° C. The hybridization stringency is preferably equivalent to 0.5×SSC and 55° C., more preferably equivalent to 0.1×SSC and 55° C., and most preferably equivalent to 0.1×SSC and 60° C.

Another aspect of the present invention provides a method for contraception in an animal, comprising immunizing the mammal with an ISP protein or a nucleic acid encoding an ISP protein. The animal is preferably a mammal and most preferably human. The ISP protein has a sequence identity with SEQ ID NO:3 or NO:4 of preferably at least about 50%, more preferably at least about 60%, yet more preferably at least about 70%, still more preferably at least about 80%, and most preferably at least about 90%. In particular, the protein may comprise SEQ ID NO:3 or NO:4. The ISP protein may be a fusion protein. Preferably, a fragment of an ISP protein is used for immunization. The fragment is at least about 10 amino acids, preferably at least about 20 amino acids, more preferably at least about 30 amino acids, yet more preferably at least about 50 amino acids, still more preferably at least about 75 amino acids, and most preferably at least about 100 amino acids in length. The fragment may be part of a fusion protein or co-administered with a carrier to elicit an immune response. Optionally, an adjuvant is also administered to enhance the immunization efficiency.

Another aspect of the present invention provides an antibody that recognizes at least one epitope of ISP1 or ISP2. The antibody may be monoclonal or polyclonal. The antibody typically has a high affinity for an ISP protein, and the Kd is preferably less than about 100 nM, more preferably less than about 30 nM, yet more preferably less than about 10 nM, and most preferably less than about 3 nM.

Also provided is a pharmaceutical composition comprising an ISP protein, a nucleic acid encoding an ISP protein, or a fragment of the ISP protein or nucleic acid. The composition may also comprise an adjuvant, a pharmaceutically acceptable excipient, and/or a pharmaceutically acceptable carrier. The ISP protein has a sequence identity with SEQ ID NO:3 or NO:4 of preferably at least about 50%, more preferably at least about 60%, yet more preferably at least about 70%, still more preferably at least about 80%, and most preferably at least about 90%. In particular, the protein comprises SEQ ID NO:3 or NO:4.

Another aspect of the present invention provides a method for contraception in an animal, comprising administering to the mammal an effective amount of an inhibitor of ISP1 or ISP2 under conditions that result in contraception. The animal is preferably a mammal and most preferably human. The inhibitor may be, for example, an antibody or an antisense oligonucleotide. Accordingly, also provided is a pharmaceutical composition comprising an inhibitor of ISP1 or ISP2.

A further aspect of the present invention provides a method for screening for inhibitors of ISP1 or ISP2, comprising providing an assay for ISP1 or ISP2 activity, determining the effect of a candidate compound on ISP1 or ISP2 activity in the assay, and identifying an inhibitor as a candidate compound capable of inhibiting ISP1 or ISP2 activity. Preferably, the inhibitor thus identified is useful in contraception.

Another aspect of the present invention provides a method for diagnosing infertility of an animal, comprising providing an assay for ISP1 or ISP2 activity/level, providing a biological sample from the animal, subjecting the biological sample to the assay, and diagnosing the animal as having infertility if ISP1 or ISP2 activity/level is low. The animal is preferably a mammal and most preferably human.

Another aspect of the present invention provides a method for treating or ameliorating infertility, comprising providing an effective amount of an ISP protein or a nucleic acid encoding an ISP protein to an animal. The animal is preferably a mammal and most preferably human. The ISP protein has a sequence identity with SEQ ID NO:3 or NO:4 of preferably at least about 50%, more preferably at least about 60%, yet more preferably at least about 70%, still more preferably at least about 80%, and most preferably at least about 90%.

Another aspect of the present invention provides a method for enhancing implantation of a cultured embryo comprising contacting the cultured embryo with an ISP protein prior to placement of the cultured embryo in the uterus of a female animal. The animal is preferably a mammal and most preferably human. The ISP protein has a sequence identity with SEQ ID NO:3 or NO:4 of preferably at least about 50%, more preferably at least about 60%, yet more preferably at least about 70%, still more preferably at least about 80%, and most preferably at least about 90%.

Similarly, the present invention also provides embryos that have been treated with an ISP protein or nucleic acid. The treated embryos can be used, for example, in infertility treatments to enhance the success rate of such treatments.

### BRIEF DESCRIPTION OF THE DRAWINGS

FIG. 1: Identification of the ISP1 cDNA from mouse implantation site RNA.

(a) Active site RT-PCR of E6.5 embryo/deciduum RNA using degenerate primers to His and Ser conserved regions of serine proteinases generates three fragments at 400-500 bp.

(b) Northern analysis of E6.5 embryo/deciduum poly(A) +RNA reveals a mRNA species of 1.3 kb which hybridizes with ISP1 cDNA.

FIG. 2: Murine ISP2 gene expression during implantation and embryogenesis.

(a) Northern analysis of E6.5 embryo/deciduum poly(A) +RNA reveals a mRNA species of 1.3 kb which hybridizes with ISP2 cDNA.

(b) ISP2 (upper panel) and GAPDH (control) expression in embryogenesis detected by RT-PCR. ISP2 gene expression in 6.5 day implantation sites (1) but not in 8.5 day (2) and 11.5 day (3) pregnancies. ISP2 gene expression in placental RNA from 11.5 day (4) and 13.5 (5) pregnancies, and 13.5 day embryo (6). ISP2 expression is not detected in hatching (7) or outgrowing (8) blastocysts.

FIG. 3: Nucleic acid sequence of the mouse ISP1 cDNA (SEQ ID NO:1)

FIG. 4: Nucleic acid sequence of the mouse ISP2 cDNA (SEQ ID NO:2)

FIG. 5: Predicted amino acid sequence of ISP1 (SEQ ID NO:3) and alignment with related serine proteinases.

Identical amino acids are marked by black boxes, conservative substitutions by grey boxes. Arrows indicate predicted pre- and pro- cleavage sites. The His and Ser active site consensus sequences are underlined.

FIG. 6: Predicted amino acid sequence for ISP2 (SEQ ID NO:4) and alignment with related serine proteinases.

Identical amino acids are marked by black boxes, conservative substitutions by grey boxes. Arrows indicate predicted pre- and pro- cleavage sites. The His and Ser active site consensus sequences are underlined.

FIG. 7: Dendrogram showing the relationship between representative serine proteinases.

The ISPs are a distinct branch of the S1 proteinase superfamily that diverged from the elastase/chymotrypsin and mast cell proteinase clusters at approximately the same time.

FIG. 8: ISP1 expression in pre-implantation embryos detected by RT-PCR.

(a) ISP1 expression in blastocysts undergoing hatching (1) or outgrowth in vitro (2).

(b) ISP1 expression in pre-implantation embryos collected as zygotes (3), morulae (4), or blastocysts (5). ISP1 expression in implantation sites (6).

(c) ISP1 expression in day 3.0 blastocysts (7); in blastocysts treated with antisense oligodeoxynucleotide (8), in blastocysts treated with control oligodeoxynucleotide (9). GAPDH is used as a control.

FIG. 9: ISP1 gene expression in morulae and blastocysts.

Morulae (a, b) and blastocysts (c, d) were stained using whole mount in situ hybridisation using sense (control) (a, c) and antisense (b, d) ISP1 probes.

FIG. 10: Inhibition of blastocyst hatching and strypsin histochemical staining with ISP1 antisense oligodeoxynucleotides

(a) Control oligodeoxynucleotide-treated blastocysts can hatch. As the zona thins the blastocyst emerges through a rupture which forms on the abembryonic pole. The black arrow indicates a blastocyst that is hatching; the white arrow indicates an empty cask after hatching.

(b) Antisense oligodeoxynucleotide-treated blastocysts cannot hatch. Shown are degenerated embryos one day after failing to hatch. Note that the zona is thickened.

(c) One day earlier than (b) showing that these embryos develop until they press against the zona. The black arrows show the very thin zonae that form when blastocysts are fully expanded.

(d) Control oligodeoxynucleotide-treated blastocysts showing normal strypsin activity staining concentrated at the abembryonic pole (white arrow).

(e) Antisense oligodeoxynucleotide-treated blastocysts display little strypsin activity at the abembryonic pole (white arrow).

FIG. 11: Inhibition of blastocyst hatching in a time dependant manner by SS1 oligodeoxynucleotide (control) or antisense AS1 oligodeoxynucleotide (experimental). Water (blank) is used as an additional control.

FIG. 12: Inhibition of blastocyst outgrowth with ISP1 antisense oligodeoxynucleotides.

(a) Prehatched blastocysts treated with control oligodeoxynucleotide invade normally into extracellular matrix.

(b) Enlarged photograph of a control blastocyst, showing invading trophoblasts (arrow).

(c) Prehatched blastocyts treated with antisense oligodeoxynucleotide fail to invade into extracellular matrix.

(d) Enlarged photograph of a blastocyst from (c), which did not invade, showing that invading trophoblasts are absent. The refraction of light that is noticed (arrow) is from the extracellular matrix previously laid down on these dishes.

FIG. 13: Expression of ISP2 mRNA in murine endometrial glands during implantation, shown by in situ hybridization of sagittally sectioned uteri from pregnant and virgin dams. Strong signal is observed distally in E7.5 (a) and E.8.5 (b) sites, and between implantation sites at E6.5 (c). ISP2 mRNA is not detected in virgin uterus (d), or uterus from E2.5 (e) or 3.5 (f) pregnancies, but is first observed in uterus from E4.5 (g) and E5.5 (h) pregnancies.

FIG. 14: Decidualization-independent ISP2 gene expression in pseudo-pregnant uterus. After priming with progesterone and estrogen, one uterine horn of the mouse was injected with sesame oil to induce decidualization. ISP2 gene expression was observed in both the decidualized (a) and non-decidualized (b) uterine horn of pseudo-pregnant females.

FIG. 15: Uterine ISP2 mRNA expression, as shown by in situ hybridization, in hormone-treated ovariectomized mice.

Pregnant dams were ovariectomized, treated immediately (a, b, c) or after a two week recovery period (d, e, f) with combinations of progesterone and/or estrogen and monitored for uterine ISP2 gene expression. ISP2 mRNA is not detected in the endometrial glands of mice that did not receive hormone treatment (a) or those treated with estrogen alone (c). ISP2 mRNA is detected in endometrial glands of mice treated with progesterone during delayed implantation (b). After ovariectomy and prolonged absence of progesterone, ISP2 gene expression is induced by progesterone treatment (d) but not with estrogen (f). The expression induced by progesterone is not significantly altered by the additional administration of estrogen (e).

FIG. 16: ISP2 mRNA is not detected uteri of pregnant and pseudo-pregnant mice that are treated with RU486 treatment, as shown by in situ hybridization.

(a) Strong ISP2 mRNA staining in the vehicle-treated (oil) pregnant uterus.

(b) No ISP mRNA staining in the smaller, RU486-treated pregnant uterus.

(c) ISP2 mRNA staining is moderate in vehicle-treated (oil) pseudopregnant uterus.

(d) No ISP2 mRNA staining in pseudopregnant RU486-treated uterus.

FIG. 17: GST Fusion Proteins of ISP1 and ISP2. (A) Regions of ISP1 (line above amino acid sequences) and ISP2 (line below amino acid sequences) were cloned into pGEX-2T vector and synthesis of the fusion proteins was induced by isopropyl β-D-thiogalactopyranoside. The fusion proteins were analyzed using polyacrylamide gel electrophoresis as shown in (B).

FIG. 18: Genomic sequence of ISP1 (mouse; SEQ ID NO:25). Sequences of the exons are underlined and bolded, and the start codon (ATG) and stop codon (TAG) of translation are darkened.

FIG. 19: Genomic sequence of ISP2 (mouse; SEQ ID NO:26). Sequences of the exons are underlined and bolded, and the start codon (ATG) and stop codon (TGA) of translation are darkened.

FIG. 20: Alignment of the predicted amino acid sequences for ISP2 (SEQ ID NO:4), human ISP2 (hISP2; SEQ ID NO:27) and ISP1 (SEQ ID NO:3). Identical amino acids are marked by black boxes, conservative substitutions by grey boxes. Arrows indicate predicted pre- and pro- cleavage sites. The active site consensus sequences for histidine and serine proteases are underlined and indicated by (His) and (Ser), respectively. The X's in the hISP2 sequence represent residues at the intron-exon boundaries that are ambiguous.

FIG. 21: cNDA sequence of human ISP2 (SEQ ID NO:34).

### DETAILED DESCRIPTION OF THE INVENTION

This invention provides two novel serine proteinases that are important for female fertility, particularly in the process of hatching and implantation. These proteinases, as well as the nucleic acids, fragments, analogs, and/or inhibitors thereof, can be used to modulate hatching, implantation and female fertility in general.

Prior to describing the invention in further detail, the terms used in this application are defined as follows unless otherwise indicated.

Definitions

“ISP1” is a protein having the sequence of SEQ ID NO:3.

“ISP2” is a protein having the sequence of SEQ ID NO:4.

An “Implantation Serine Proteinase protein”, or “IS P protein”, is a protein that possesses at least one biological activity of ISP1 or ISP2, as well as a substantial sequence identity with mouse ISP1 (SEQ ID NO:3) or ISP2 (SEQ ID NO:4). ISP proteins include, for example, mutants, variants and derivatives of ISP1 or ISP2. The ISP protein further preferably has a substantial sequence identity with regions of SEQ ID Nos:3 or 4 that are less similar with the other proteinases. These regions are areas that are not the IVGG, His active site, or Ser active site, and in particular amino acid number 80 to the C-terminus of SEQ ID NO:3 or SEQ ID NO:4.

Biological activities of ISP1 and ISP2 include the biological activities disclosed herein, such as proteinase activity, hatching activity, pregnancy-promoting activity, and the ability to be recognized by an antibody raised against ISP1 or ISP2. The proteinase activity is the activity to cleave a protein into at least two fragments, each of which fragments has at least one amino acid. The hatching activity is the participation of a protein in the process of hatching, which can be determined according to this disclosure or other established methods in the art. By way of examples, the hatching activity of strypsin was assayed as disclosed in Perona and Wassarman, 1986. A protein has a pregnancy-promoting activity if it enhances the chance of pregnancy, or if an inhibitor of the protein reduces or eliminates the chances of pregnancy.

A “substantial sequence identity” is a sequence identity of at least about 40% at either the nucleotide or amino acid level. Typically, the percentage of sequence identity is at least approximately one of the following: 45, 50, 55, 60, 65, 70, 75, 80, 85, 90, and 95. The sequence identity is preferably at least about 50%, more preferably at least about 65%, still more preferably at least about 75%, yet more preferably at least about 85%, even more preferably at least about 90%, and most preferably at least about 95%. Alternatively, a nucleic acid shares a substantial sequence identity with another nucleic acid if they hybridize to each other under a hybridization condition with a stringency equivalent to 0.5×SSC and 50° C. The hybridization stringency is preferably equivalent to 0.5×SSC and 55° C., more preferably equivalent to 0.0×SSC and 55° C., and most preferably equivalent to 0.1×SSC and 60° C. If a protein has more than one subunit, it is sufficient that any one subunit has a substantial sequence identity with ISP1 or ISP2 for the protein to be deemed as having a substantial sequence identity with ISP1 or ISP2, respectively. The degree of sequence identity, either at the amino acid or nucleotide level, can be determined with any algorithm, preferably BLAST. In addition, an ISP protein preferably has the sequences that qualify as His and/or Ser protease active sites, such as LTAAHC (SEQ ID NO:5) and/or GDSGGPL (SEQ ID NO:6).

A “variant” of ISP1 or ISP2 is a naturally-occurring ISP protein, including, for example, allelic variants of ISP1 or ISP2, naturally-occurring ISP proteins isolated from a species other than mice, and other naturally-occurring mouse ISP proteins which are not ISP1 or ISP2.

A “mutant” of an ISP protein is an ISP protein that is generated by recombinant DNA techniques by changing the amino acid sequence of the original ISP protein.

A “derivative” ISP protein is a chemically-modified ISP protein in which at least one side chain of an amino acid of an ISP protein has been chemically modified.

A “recombinant protein” is a protein expressed from an exogenously introduced nucleic acid.

A nucleic acid “encoding” or “coding for” a protein if the nucleotide sequence of the nucleic acid can be translated to the amino acid sequence of the protein. The nucleic acid, however, does not have to contain an actual translation start codon or termination codon.

The term “contraception” means a reduction or elimination of the chance of pregnancy.

The term “immunizing” means introducing antigen into a mammal under conditions wherein an immune response against the antigen is elicited. The immune response includes, but is not limited to, antibody production and cellular immunity. In the process of immunization, a protein antigen may be introduced as a protein or as a nucleic acid encoding the protein.

A “fusion protein” is a recombinant protein comprising regions derived from at least two different proteins.

An “antibody” is a protein molecule that reacts with a specific antigen and belongs to one of five distinct classes based on structural properties: IgA, IgD, IgE, IgG and IgM.

The term “infertility” means the inability or difficulty of an animal to become pregnant.

An animal is “pregnant” if an embryo is implanted in the uterus of the animal.

A “biological sample” is a sample collected from a biological subject, such as an animal, plant or microorganism.

A “mammal” is any mammalian animal. The mammal is preferably a primate, rodent, canine, feline, or domestic livestock. In particular, the mammal may be a human, dog, cat, cattle, sheep, goat, mouse, rat, or rabbit.

An “effective amount” is an amount which is sufficient to achieve the intended purposes. For example, an effective amount of an ISP protein for the purpose of contraception is an amount sufficient to result in a reduction or elimination of the chance of pregnancy in the animal receiving the ISP protein. The effective amount of a given therapeutic agent will vary with factors such as the nature of the agent, the route of administration, the size and species of the animal to receive the therapeutic agent, and the purpose of the administration. The effective amount in each individual case may be determined empirically by a skilled artisan according to established methods in the art.

“Treating or ameliorating” means the reduction or complete removal of the symptoms of a disease or medical condition.

Cloning of ISP1 and ISP2

To identify novel serine proteinases important for implantation, degenerate primers to the conserved His and Ser regions of the active site of known serine proteinases were designed. These primers also contained nucleotide recognition sites for restriction endonucleases, which allowed directional cloning into a plasmid vector. mRNA was isolated from embryos and implantation sites, and RT-PCR was performed using conditions which optimized the synthesis of PCR products of the appropriate size for a serine proteinase (about 500 nucleotides in length), while minimizing other background bands. The PCR products of the appropriate size were isolated from a gel, cloned into plasmid vector such as pBluescriptTM and sequenced, for example by cycle sequencing.

Computer analysis of these sequences, such as with a BLAST™, was performed to identify the novelty of the isolated sequences. mRNA expression patterns of the novel proteinases were examined to determine whether the clones are involved in hatching, implantation or early murine development. Full-length cDNA clones of the novel genes of interest were obtained by screening a cDNA library, sequenced, and determined to be full length if they contain the appropriate recognition sequences for the initiation of translation, a start Met codon and a poly(A)⁺ tail separated by an open reading frame.

Two novel serine proteinases were identified, which are expressed during hatching and implantation, and are thus named Implantation Serine Proteinase (ISP) 1 and 2. The nucleic and amino acid sequences of ISP1 and ISP2 demonstrate that these proteins have hallmark signatures of tryptases: the His, Ser and Asp active site regions, the N-terminal IVGG sequence, and a homology to trypsin. However, maximum parsimony analysis indicates that they represent a distinct lineage of the S1 superfamily, having first diverged from the mast cell proteinase and elastase/chymotrypsin clusters at approximately the same time.

Previously, Vu et al. (1997) used serine proteinase active site RT-PCR of RNA derived from preimplantation embryos and identified hepsin, a membrane-associated serine proteinase that is also expressed in kidney and liver. Gene disruption studies demonstrated that this serine proteinase is not the mammalian hatching enzyme (Wu et al., 1998). ISP1 or ISP2 was not identified by Vu et al. Similarly, our search did not identify hepsin.

ISP1 Function

PCR analysis using primers that are specific for ISP1 demonstrated that ISP1 mRNA is expressed during blastocyst hatching and outgrowth. ISP1 is expressed in both blastocysts and the uterine endometrial glands. This uterine ISP1 expression is regulated by progesterone, which plays an important role in pregnancy.

Two antisense oligodeoxynucleotides targeted against ISP1 interfered with in vitro blastocyst hatching in a concentration- and time-dependant manner. Some blastocysts were able to escape hatching arrest, presumably due to degradation of oligodeoxynucleotide within culture media. If the oligodeoxynucleotides were administered in fresh medium over time, a prolonged interference on hatching was observed, and fresh oligodeoxynucleotide medium was required to affect blastocyst outgrowth over 5 days. Further, if the oligodeoxynucleotides were allowed to dissipate in medium, blastocysts were able to escape the block on outgrowth. The observation that blastocysts can escape hatching and outgrowth arrest indicates that the antisense oligodeoxynucleotides are not toxic, and we noted that blastocysts did not die as a consequence of antisense oligodeoxynucleotide treatment. However, embryonic death does occur when blastocysts fail to hatch or outgrow after a period of time.

Antisense oligodeoxynucleotides that are targeted against the initiation codon of mRNAs inhibit translation and result in the degradation of target transcripts (Schlingensiepen and Brysch, 1992). Indeed, antisense oligodeoxynucleotides against ISP1 specifically blocked the accumulation of ISP1 mRNA in blastocysts eight hours after treatment. This blockage is transient, as ISP1 mRNA levels returned almost to normal after 24 hours of treatment.

This expression pattern, the fact that ISP1 antisense oligonucleotides interfere with hatching, as well as the similarity of tryptases to trypsin, indicates that ISP1 may encode the trypsin-like activity involved in blastocyst hatching, strypsin (Perona and Wassarman, 1986). Since the ISP1 gene is expressed throughout the blastocyst and strypsin activity is extracellularly localized to the distal pole of the blastocyst, ISP1 protein is likely either recruited to the abembryonic pole for activity, or is preferentially translated in apical trophoblasts.

The predicted molecular weight of ISP1 (˜27,000 Da) is considerably smaller than the native molecular weight of strypsin (74,000 Da), which suggests that if ISP1 is strypsin, it must multimerize for activity. This is consistent with the observation that tryptases, including mouse mast cell proteinases, multimerize for activity and are assembled with the assistance of heparin sulfate proteoglycans (Lindstedt et al., 1998; Huang et al., 2000). Indeed, the abembryonic pole of the blastocyst is rich in heparin sulfate proteoglycan (Farach et al., 1987), and heparinase digestion has demonstrated that this heparin sulfate is required for blastocyst attachment and outgrowth (Farach et al., 1987). Similarly, the actions of maternal heparin sulfate binding-EGF in stimulating blastocyst hatching and outgrowth may be explained by the pH dependence of tryptase activation (Lindstedt et al., 1998; Huang et al., 2000) and the changes in ion flux that occur downstream of HB-EGF binding to the ErbB4 receptor (Wang et al., 2000).

Based on the molecular weight of ISP1 and strypsin, ISP1 is expected to form a tetramer. We investigated this possibility by using the Swiss protein modeling algorithms (SWISS-MODEL (Peitsch et al., 2000;

http://www.expasy.org/swissmod/SWISS-MODEL.html) and Rasmol

(http://www.umass.edu/microbio/rasmol/). Thus, ISP1 was layered on the tetrameric scaffold of a previously defined structure of human beta tryptase, and the results indicate that ISP1 is capable of forming tetramers.

Our antisense RNA experiments have helped to clarify the mechanism of hatching. During these experiments, zona thinning occurred in conjunction with blastocyst growth. When blastocysts died within the zona, regression of the embryo resulted in a reversal of the zona thinning. This observation confirms the hypothesis that zona thinning is dependent, at least in part, upon blastocyst expansion. Blastocyst expansion and zona thinning may play an important regulatory role by ensuring that hatching occurs at an appropriate time, perhaps by exposing specific proteolytic sites in this proteinaceous sheath. After Mintz (Mintz, 1972) and Pinsker et al. (Pinsker et al., 1974) first suggested the enzyme responsible for hatching might also be an implantation initiation factor, Gonzales and Bavinster (1995) predicted that the enzyme responsible for focal hatching in vitro might really be the enzyme responsible for facilitating blastocyst attachment and invasion. The present invention confirmed this additional role for ISP1 in facilitating implantation competence.

The abembryonic pole of the blastocyst becomes competent to attach and invade into extracellular matrix in vitro, and this competence occurs as a function of localized heparin sulfate proteoglycan and the action of heparin binding EGF (Farach et al., 1987, Das et al., 1994). Without being limited to any theory, ISP1 may function in connecting embryo hatching to the initiation and establishment of implantation competence at the abembryonic pole of the blastocyst. Historically, hatching and outgrowth have been viewed as unrelated molecular phenomena. While serine proteinase inhibitors have been shown to affect both hatching (Perona and Wassarman, 1986) and outgrowth (Kubo et al., 1981; Behrendtsen et al., 1992), these studies have focused on the respective roles of strypsin in hatching and uPA in outgrowth. Most, if not all of the inhibitors used in this study, including bis[5-amido-2-benzimidazole], are effective against tryptases (Compton et al., 1998; Sanderson, 1999). The present invention indicates that the actions of these inhibitors in affecting outgrowth may have been directed to ISP1, as well as its capability of degrading the extracellular matrix, and ISP1 are indispensable for both hatching and blastocyst outgrowth.

In addition to degrading extracellular matrix in blastocyst outgrowth, ISP1 (strypsin) may also participate indirectly in ECM degradation through the activation of other proteinases such as MMP9, which is activated by tryptases in vivo (Lohi et al., 1992; Keski-Oja et al., 1992). While removal of the zona barrier has long been viewed as the critical first step in implantation, our results demonstrate that the role of the hatching proteinase in implantation may be more active than passive. Based on its early expression, ISP1 (strypsin) may be a lynch pin in the cascade of proteinase activity during implantation.

The role of the hatching proteinase in facilitating embryo attachment and outgrowth also explains why assisted hatching procedures performed in fertility clinics have failed to promote the successful implantation of human embryos (see De Vos and Van Steirteghem, 2000 for review). Indeed, embryos from women of advanced age frequently fail to hatch in vitro and may be devoid of hatching enzyme activity (Bider et al., 1997). The ISP1 gene may thus be used as an important diagnostic tool in human fertility, while compositions comprising the ISP1 protein may be used to improve assisted reproduction.

ISP2 Function

We found that during gestation, the ISP2 gene is expressed predominantly during implantation, although residual expression is observed in the developing placenta. Unlike ISP1, the ISP2 gene is not expressed in the pre-implantation embryo. Instead, in situ hybridization experiments demonstrate that ISP2 gene expression is observed in endometrial gland epithelium throughout the peri-implantation period (days 4.5 to 8.5). During implantation, ISP2 gene expression initially occurs in glands throughout the decidua, including regions proximal to the embryo, but it becomes restricted when the glands diminish in size and move to the periphery of the uterine crypt during deciduum regression and placentation.

In situ hybridization experiments reveal that ISP2 gene expression is regulated by progesterone. Hybridization of ISP2 mRNA in glandular epithelium lying between implantation sites suggests that ISP2 gene expression might not be dependent upon the presence of the embryo. This is confirmed when oil induced deciduomas are established in hormonally treated, pseudopregnant females. ISP2 mRNA is detected within the glands of non-decidualized control horns. Further investigation using ovariectomy, and models of delayed implantation, demonstrated that ISP2 gene expression is dependent only upon progesterone administration. Estrogen had no effect either on its own or in combination with progesterone. In the presence of the anti-progestin, RU486, ISP2 gene expression was abrogated in both pregnancy and pseudo-pregnancy. Accordingly, glandular ISP2 gene expression is positively regulated by progesterone.

A key feature of successful implantation is the synchrony between embryonic and endometrial development. This synchrony is achieved through timely preparation regulated first by hormones, and after blastocyst hatching by cytokine signaling between the endometrium and the embryo. Only on day 4 of pregnancy, as progesterone levels rise, does the glandular epithelium differentiate and become secretory (Duc-Goiran et al., 1999; Paria et al., 1999). Our in situ hybridization experiments demonstrate that ISP2 mRNA is not detected at stages that precede the endometrial gland secretory phase. Therefore, ISP2 secretion into the glandular and uterine lumen may occur as a consequence of progesterone induced epithelial differentiation.

The endometrial gland acts as a “command center” in pregnancy, sending and receiving cytokine dispatches that support implantation. Animals devoid of endometrial glands cannot support pregnancy (Gray et al., 2000). Leukemia Inhibitory Factor (LIF), for example, is secreted from the endometrial gland into the uterine lumen, where it is thought to interact with luminal LIF receptors and result in the presentation of EGF receptors that are necessary for apposition of the embryo (Song et al., 2000). The LIF gene is not expressed following RU486 administration (Danielsson et al., 1997; Ghosh et al., 1998, Liu et al., 1999), as observed for ISP2.

RU486 has a profound effect on preventing the differentiation of secretory glandular epithelium, which likely accounts for its effect on LIF expression and in preventing implantation (Greb et al., 1999). LIF secretion is distinct from ISP2 in that it is also estrogen-dependent (Song et al., 2000). While estrogen appears to co-ordinate LIF's expression during the “window of implantation”, a morphologically normal endometrial gland is necessary for secretion into the lumen. This role of progesterone in generating a fully functional endometrial gland explains why in delayed implantation, progesterone priming is required prior to the estrogen pulse. Since ISP2 gene expression is independent of the estrogen spike and occurs during the progesterone priming-phase, ISP2's first proteolytic role precedes implantation.

ISP1 and ISP2 are the only known serine proteinases that are expressed in the endometrial gland. Matrix metalloproteinase MMP9 is expressed in glandular epithelium during implantation and found in uterine luminal fluid (Jeziorska et al., 1996), and is presumed to participate in the ECM remodeling that occurs during implantation. Since MMP9 is activated by tryptases in vivo (Lohi et al., 1992; Keski-Oja et al., 1992), ISP2 could potentially activate MMP9. In addition, a direct role for ISP2 in matrix remodeling is also possible.

Based on the emerging role of tryptases in effecting extracellular signaling (Mirza et al., 2000), ISP1 and ISP2 function within the embryo and uterus may not be restricted to matrix remodeling. Recently, serine proteinases have been found to have multiple roles in extracellular signaling. Mast cell tryptases, in particular, have recently been implicated as paracrine factors, having been recognized as mediators of cellular mitogenesis and differentiation through the cleavage of tethered ligands on a new class of G protein-linked receptor that is proteinase activated (Mirza et al., 2000). Likewise, related serine proteinases such as plasmin and elastase have been found to participate in signaling either by releasing of tethered cytokines or shedding cytokine receptors (Taipale and Keski-Oja, 1997; Muller-Newen et al., 1996). Based on the central role that the endometrial gland plays in dispatching and receiving important implantation cytokine signals (i.e. LIF, CSF, IGF) under hormonal control, ISP2 might also be playing a role in modulating important extracellular signals that orchestrate implantation. For example, bound forms of LIF, CSF and IGF have been identified in pregnancy (Rathjen et al., 1990, Pampfer et al., 1991, Rutanen, 2000), as have soluble forms of LIF receptor, gp130 and LIF-receptor alpha-chain (Zhang et al., 1998). Secretion of ISP2 into the endometrial gland lumen may be associated with the shedding of these cytokines and/or receptors.

Without being limited to a theory, we believe that ISP2 functions as a uterine proteinase that is involved in the degradation of the zona prior to implantation, and it also has an additional role in mediating cytokine signaling during implantation.

Uterine “lysins” have been suggested to be important for promoting zona thinning (Montag et al., 2000). An emerging literature has suggested that the “focal” hatching, which occurs in vitro, is distinct from hatching in utero, where the zona appears to “dissolve” after thinning (Gonzales and Bavister, 1995; Montag et al., 2000). As hatching occurs approximately one day earlier in utero than in vitro, Gonzales and Bavinster (1995) have described in vitro hatching as an artifact characterized by the absence of a uterine “lysin proteinase”. Accordingly, it has been suggested that a distinct “lysin” protein occurs in lumenal fluid prior to implantation. Evidence suggests that a hormonally regulated proteinase associated with uterine secretions contributes to hatching (Orsini and McLaren, 1967; Joshi and Murray, 1974; Rosenfeld and Joshi, 1981) and that this proteinase is progesterone regulated (Denker, 1977). However, it does not require the presence of an embryo within the uterus (Orsini and McLaren, 1967). Based on the striking similarity in expression profiles between “lysin” and ISP2, we believe that the ISP2 gene encodes this uterine “lysin proteinase”

ISP2, like ISP1, is capable of forming tetramers in silica when analyzed by the protein modeling algorithms described above. Moreover, ISP1 and ISP2 can form heterotetramers with a considerably higher stability than either homotetramer. We further discovered that ISP1 is expressed in endometrial glands in a temporal and spatial pattern similar to that of ISP2. In fact, Western blot analyses suggest that ISP1 and ISP2 form a heteromultimer (most likely heterotetramer) in the uterus. Without wishing to be limited to theory, we believe that ISP1 and ISP2 that are expressed in the endometrial glands interact with each other in the uterine lumen and facilitate hatching from outside the embryo. In addition, embryonic ISP1 also enhances the interaction between hatched blastocyst and the uterine wall. The present invention thus provides both homomers of ISP1 or ISP2, as well as heteromers of ISP1 and ISP2, in the use of hatching, implantation and infertility treatment.

Compositions

The present invention provides novel Implantation Serine Proteinase (ISP) proteins and nucleic acids encoding the ISP proteins. The ISP proteins possess at least one biological activity of ISP1 or ISP2, as well as a substantial sequence identity with ISP1 (SEQ ID NO:3) or ISP2 (SEQ ID NO:4). Biological activities of ISP1 and ISP2 are described herein, including proteinase activity, hatching activity, pregnancy-promoting activity, and the ability to be recognized by an antibody raised against ISP1 or ISP2. The proteinase activity is the activity to cleave a protein into at least two fragments, each of which has at least one amino acid. The hatching activity is the participation of a protein in the process of hatching, which can be determined according to the this disclosure or established methods in the art. Preferably, the hatching activity is determined by adding antisense nucleic acids, antibodies, or inhibitors of an ISP protein to a hatching system, or by knock-out experiments.

An ISP protein shares a substantial sequence identity with ISP1 or ISP2. The ISP proteins encompass insertional, deletional, and substitutional variants or mutants of ISP1 and ISP2. These mutants ordinarily are prepared by site specific mutagenesis of nucleotides in the DNA encoding ISP1 or ISP2, by which DNA encoding the mutant is obtained, and thereafter expressing the DNA in recombinant cell culture. However, mutant ISP protein fragments having up to about 100 to 150 amino acid residues may be prepared conveniently by in vitro synthesis.

The ISP protein mutants typically exhibit the same qualitative biological activity as naturally occurring ISP proteins. However, the ISP proteins that are not capable of exhibiting qualitative biological activity similar to native ISP proteins (except for antibody cross-reactivity) may nonetheless be useful as reagents in diagnostic assays for ISP proteins or antibodies to ISP proteins. Moreover, when insolubilized in accordance with known methods, they may be used as agents for purifying anti-ISP protein antibodies from antisera or hybridoma culture supernatants. Furthermore, they may be used as immunogens for raising antibodies to ISP proteins or as a component in an immunoassay kit (labeled so as to be a competitive reagent for native ISP proteins or unlabeled so as to be used as a standard for the ISP protein assay) so long as at least one ISP1 or ISP2 epitope remains active in these analogs.

In addition, an ISP protein may be an antagonist of ISP1 or ISP2. An antagonist may be identified, for example, as a protein that can inhibit the activity of ISP1 or ISP2 in a biological assay for ISP1 or ISP2.

While the site for introducing an amino acid variation may be predetermined, the mutation, per se, need not be predetermined. For example, in order to optimize the performance of a mutation at a given site, random or saturation mutagenesis (where all 20 possible residues are inserted) is conducted at the target codon and the expressed ISP protein mutant is screened for the optimal combination of desired activities. Such screening is within the ordinary skill of the art.

Amino acid insertions will usually be on the order of from about one to about ten amino acid residues; substitutions are typically introduced for single residues and deletions will range from about one to about thirty residues. Deletions or insertions preferably are made in adjacent pairs. That is, a deletion of two residues or insertion of two residues. Substitutions, deletions, insertions or any combination thereof may be introduced to a single mutant.

Insertional mutants of a native ISP protein are those in which one or more amino acid residues extraneous to native ISP proteins are introduced into a predetermined site in the target ISP protein. Commonly, insertional variants are fusions of heterologous proteins or polypeptides to the amino or carboxyl terminus of the ISP protein. Such mutants are referred to as fusion proteins of the ISP protein and a polypeptide containing a sequence which is other than that which is normally found in the ISP protein at the inserted position.

Immunologically active ISP protein derivatives and fusions comprise an ISP protein and a polypeptide containing a non-ISP protein epitope. Such immunologically active derivatives and fusions of ISP protein are within the scope of this invention. The non-ISP protein epitope may be any immunologically competent polypeptide, i.e., any polypeptide which is capable of eliciting an immune response in the animal in which the fusion is to be administered, or which is capable of being bound by an antibody raised against the non-ISP protein polypeptide.

Substitutional mutants are those in which at least one residue of ISP1 or ISP2 has been removed and a different residue inserted in its place. Novel amino acid sequences as well as isosteric analogs (amino acid or otherwise) are included within the scope of this invention.

Some deletions, insertions and substitutions will not produce radical changes in the characteristics in the ISP protein molecule. However, while it is difficult to predict the exact effect of the substitution, deletion or insertion in advance of doing so, for example, when modifying an immune epitope on the ISP protein protein, one skilled in the art will appreciate that the effect can be evaluated by routine screening assays. For example, a change in the immunological character of an ISP protein protein, such as affinity for a given antibody, can be measured by a competitive-type immunoassay. Modifications of protein properties such as redox or thermal stability, hydrophobicity, susceptibility to proteolytic degradation, or the tendency to aggregate with carriers or into multimers may be assayed by methods well known to one of skill in the art.

Deletions of cysteines or other labile amino acid residues may also be desirable. For example, such deletions may increase the oxidative stability of the ISP protein. Deletion or substitution of potential proteolysis sites, e.g., Arg Arg, can be accomplished by deleting one of the basic residues or substituting one with glutaminyl or histidyl residues.

Covalent modifications of the ISP protein are included within the scope of the present invention. Such modifications are introduced by reacting targeted amino acid residues with an organic derivatizing agent that is capable of reacting with selected side chains or terminal amino acid residues. The resulting covalent derivatives of an ISP protein are useful to identify residues important for the ISP protein's biological activity, for immunoassays of the ISP protein or for preparation of anti-ISP protein antibodies for affinity purification of recombinant ISP proteins. Such modification are within the ordinary skill of the art and are performed without undue experimentation.

Also provided are fragments of an ISP protein. Fragments of an ISP protein can be used, for example, to raise antibodies, detect antibodies in a biological sample, or screen for agonists or antagonists of the ISP protein. A fragment is at least 10 amino acids long. The fragment is preferably at least about 30, more preferably at least about 50, yet more preferably at least about 100, and most preferably at least about 150 amino acids long. The fragment may be part of a fusion protein.

Further provided are nucleic acid fragments of the nucleic acids encoding ISP proteins. The fragments can be used to express ISP proteins or protein fragments. Further more, the nucleic acid fragments can be used, for example, as probes in nucleic acid analysis, primers for nucleic acid extension, or antisense nucleic acids. The fragments may be single- or double-stranded, and are at least about 15 nucleotides in length. The fragments are preferably at least about 30, more preferably at least about 50, yet more preferably at least about 100, still more preferably at least about 200, even more preferably at least about 300, and most preferably at least about 400 nucleotides in length.

The present invention also provides vectors comprising a nucleic acid encoding an ISP protein, as well as prokaryotic and eukaryotic cells comprising such vectors. Such vectors ordinarily carry a replication site, although this is not necessary where chromosomal integration will occur. Expression vectors may also include marker sequences which are capable of providing phenotypic selection in transformed cells. Expression vectors also optimally will contain sequences which are useful for the control of transcription and translation.

Expression vectors used in eukaryotic host cells will also contain sequences necessary for the termination of transcription which may affect mRNA expression. Expression vectors may contain a selection gene as a selectable marker. Examples of suitable selectable markers for mammalian cells are dihydrofolate reductase, thymidine kinase, neomycin or hygromycin.

The present invention also provides antibodies that recognize at least one epitope of ISP1 or ISP2. Antibodies to an ISP protein may be prepared in conventional fashions (Harlow et al., 1988) by injecting goats or rabbits. For example, a complete ISP protein or a peptide consisting of at least 10 amino acids similar to the ISP protein, in complete Freund's adjuvant, can be injected subcutaneously, followed by booster intraperitoneal or subcutaneous injection in incomplete Freund's adjuvant. The anti-ISP protein antibodies may be directed against one or more epitopes of an ISP protein. Monoclonal antibodies against ISP proteins can be prepared by methods known in the art (Harlow et al., 1988. The antibodies may be labeled with a marker, for example, with a radioactive or fluorescent marker. It is contemplated that the antibodies would be labeled indirectly by binding them to an anti-goat or anti-rabbit antibody covalently bound to a marker compound.

An ISP protein, nucleic acid (including antisense nucleic acids), fragment thereof, vector or host cell can be comprised in a composition with other components. Specifically, a pharmaceutical composition is provided, which preferably comprises a pharmaceutical acceptable excipient and/or carrier. In making the compositions of this invention, the active ingredient is usually mixed with an excipient, diluted by an excipient or enclosed within such a carrier which can be in the form of a capsule, sachet, paper or other container. When the pharmaceutically acceptable excipient serves as a diluent, it can be a solid, semi-solid, or liquid material, which acts as a vehicle, carrier or medium for the active ingredient. Thus, the compositions can be in the form of tablets, pills, powders, lozenges, sachets, cachets, elixirs, suspensions, emulsions, solutions, syrups, aerosols (as a solid or in a liquid medium), ointments containing, for example, up to 10% by weight of the active compound, soft and hard gelatin capsules, suppositories, sterile injectable solutions, and sterile packaged powders.

Some examples of suitable excipients include lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, sterile water, syrup, and methyl cellulose. The formulations can additionally include: lubricating agents such as talc, magnesium stearate, and mineral oil; wetting agents; emulsifying and suspending agents; preserving agents such as methyl- and propylhydroxy-benzoates; sweetening agents; and flavoring agents. The compositions of the invention can be formulated so as to provide quick, sustained or delayed release of the active ingredient after administration to the patient by employing procedures known in the art.

For preparing solid compositions such as tablets, the principal active ingredient is mixed with a pharmaceutical excipient to form a solid preformulation composition containing a homogeneous mixture of a compound of the present invention. When referring to these preformulation compositions as homogeneous, it is meant that the active ingredient is dispersed evenly throughout the composition so that the composition may be readily subdivided into equally effective unit dosage forms such as tablets, pills and capsules.

The tablets or pills of the present invention may be coated or otherwise compounded to provide a dosage form affording the advantage of prolonged action. For example, the tablet or pill can comprise an inner dosage and an outer dosage component, the latter being in the form of an envelope over the former. The two components can be separated by an enteric layer which serves to resist disintegration in the stomach and permit the inner component to pass intact into the duodenum or to be delayed in release. A variety of materials can be used for such enteric layers or coatings, such materials including a number of polymeric acids and mixtures of polymeric acids with such materials as shellac, cetyl alcohol, and cellulose acetate.

The liquid forms in which the novel compositions of the present invention may be incorporated for administration orally or by injection include aqueous solutions, suitably flavored syrups, aqueous or oil suspensions, and flavored emulsions with edible oils such as corn oil, cottonseed oil, sesame oil, coconut oil, or peanut oil, as well as elixirs and similar pharmaceutical vehicles.

Compositions for inhalation or insufflation include solutions and suspensions in pharmaceutically acceptable, aqueous or organic solvents, or mixtures thereof, and powders. The liquid or solid compositions may contain suitable pharmaceutically acceptable excipients as described herein. Preferably the compositions are administered by the oral or nasal respiratory route for local or systemic effect. Compositions in preferably pharmaceutically acceptable solvents may be nebulized by use of inert gases. Nebulized solutions may be inhaled directly from the nebulizing device or the nebulizing device may be attached to a face mask tent, or intermittent positive pressure breathing machine. Solution, suspension, or powder compositions may be administered, preferably orally or nasally, from devices which deliver the formulation in an appropriate manner.

Another preferred formulation employed in the methods of the present invention employs transdermal delivery devices (“patches”). Such transdermal patches may be used to provide continuous or discontinuous infusion of the active ingredient in controlled amounts. The construction and use of transdermal patches for the delivery of pharmaceutical agents is well known in the art. See, for example, U.S. Pat. No. 5,023,252, herein incorporated by reference. Such patches may be constructed for continuous, pulsatile, or on demand delivery of pharmaceutical agents.

Compositions of the present invention for immunizing animals may also comprise an adjuvant to increase immunoprotective antibody titers or cell mediated immunity response. Such adjuvants may include, but are not limited to, Freunds complete adjuvant, Freunds incomplete adjuvant, aluminum hydroxide, dimethyldioctadecyl-ammonium bromide, Adjuvax (Alpha-Beta Technology), Inject Alum (Pierce), Monophosphoryl Lipid A (Ribi Immunochem Research), MPL+TDM (Ribi Immunochem Research), Titermax (CytRx), QS21, the CpG sequences (Singh et al., 1999), toxins, toxoids, glycoproteins, lipids, glycolipids, bacterial cell walls, subunits (bacterial or viral), carbohydrate moieties (mono-,di-, tri-, tetra-, oligo- and polysaccharide), various liposome formulations or saponins.

Other suitable formulations for use in the present invention can be found in Remington's Pharmaceutical Sciences (19^{th} Ed.).

Methods

The present invention provides methods for producing an ISP protein using a nucleic acid encoding the ISP protein. Briefly, an expression vector comprising the nucleic acid is constructed and introduced into a suitable cell, transformants are selected and cultured under conditions leading to production of the ISP protein, and the ISP protein is isolated. Suitable cells include, for example, bacterial, yeast, insect and mammalian cells.

As demonstrated herein, the ISP proteins have hatching and implantation activities, and can be used for contraception. Contraception may be achieved by immunizing an animal with an ISP protein to elicit an immune response to the ISP protein, thereby interfering with the function of the protein, which is essential for conception. Contraception may also achieved by administering an inhibitor of an ISP protein, which inhibitor is capable of inhibiting the function of the protein essential for conception. The inhibitor may be, for example, an antibody against the ISP protein or an antisense nucleic acid that can reduce the amount of the ISP protein. The inhibitor may also be a chemical compound identified by its ability to inhibit the proteinase, hatching or implantation activity of the ISP protein in drug screening. The proteinase, hatching or implantation activity of ISP proteins may be assayed according to the present disclosure or methods known in the art.

The present invention can also be used to diagnose infertility, and particularly infertility associated with low ISP protein level or activity. Thus, a biological sample may be obtained from the animal to be diagnosed and subjected to an ISP assay. An assay result of an ISP activity or level lower than the normal range would indicate that the animal has a reduced chance to become pregnant. The normal range can be obtained from a population of the same animal who are fertile. The assay can be an assay for ISP activities, such as proteinase, hatching or implantation activities, or an assay for ISP protein levels using, for example, antibodies against the ISP protein.

ISP can also be used to enhance in vitro fertilization by incubating a cultured embryo in the presence of an ISP protein before the embryo is placed in the uterus of a female animal. Such an application is within the skills of the art. For example, it has recently been shown that enzymatic treatment of the zona of human embryos with pronase before transfer to a receptive uterus dramatically increased the implantation rate of the embryos (Fong et al., 1998).

The following examples are offered to illustrate this invention and are not to be construed in any way as limiting the scope of the present invention.

### EXAMPLES

In the examples below, the following abbreviations have the following meanings. Abbreviations not defined have their generally accepted meanings.

| | |
|---|---|
| ° C. = | degree Celsius |
| hr = | hour |
| min = | minute |
| μM = | micromolar |
| mM = | millimolar |
| M = | molar |
| ml = | milliliter |
| μl = | microliter |
| mg = | milligram |
| μg = | microgram |
| PAGE = | polyacrylamide gel electrophoresis |
| rpm = | revolutions per minute |
| FBS = | fetal bovine serum |
| DTT = | dithiothrietol |
| SDS = | sodium dodecyl sulfate |
| PBS = | phosphate buffered saline |
| DMEM = | Dulbecco's modified Eagle's medium |
| α-MEM = | α-modified Eagle's medium |
| β-ME = | β-mercaptoethanol |
| EGF = | epidermal growth factor |
| PDGF = | platelet derived growth factor |
| DMSO = | dimethylsulfoxide |
| IPTG = | isopropyl β-D-thiogalactopyranoside |

### Materials and Methods

Animals and Treatments

CD1 mice were obtained at the age of 6-7 weeks from Charles River Canada (St. Constant, PQ) and maintained in a standard laboratory animal facility with controlled temperature (20° C.) and lighting (lights-on between 0700 h and 1900 h). The maintenance and treatment of the animals were in full compliance with standard laboratory animal care protocols approved by the University of Calgary's Animal Care Committee. To obtain natural pregnancies, female mice were paired with adult males and checked daily for the presence of a vaginal copulatory plug as an indication of mating. For embryo collection, day 0.5 corresponded to midday of the day a vaginal plug was detected. Pregnant dams were sacrificed on a specific embryonic day by cervical dislocation, after which, uteri and/or oviducts were surgically removed prior to isolation of embryos, either by dissection or flushing (Hogan et al., 1994). All surgical procedures were carried out after the mice were anaesthetized with an i.p. injection of Avertin (2% (w/v) tribromoethanol; Aldrich Chemical Co. Milwaukee, Wis.). Ovariectomy was performed by dorsal-lateral incision (Hogan et al., 1994). Ovariectomized mice were allowed at least 1 wk for recovery before the induction of deciduomas. All steroid hormones including RU486 were dissolved in sesame oil and injected s.c. At each stage of the experiment, control mice were used which received only oil (0. 1 ml/mouse) injections. The standard regimen for artificial induction of deciduomas (Finn, 1966) was modified to more closely mimic pseudo-pregnancy (Milligan, 1995). Here, the first progesterone treatment was started two days after exposure to oestrogen. This modified regimen consisted of 100 ng oestrogen daily starting on Day 0, and 1 mg progesterone plus 10 ng oestrogen from day three onward. Deciduomas were induced surgically on Day 5 (between 1400-1600 h) by injecting sesame oil (10 μl) into the lumen of one uterine horn from its oviductal tip. Injected and uninjected horns were collected 24, 48 or 72 hours later for histological sectioning and in situ hybridization analysis. Delayed implantation was induced and maintained by ovariectomising mice on day 3 of pregnancy, followed by administration of progesterone (2 mg/ mouse) on days 4 to 6. Subsequently, half these mice were treated with estrogen (25 ng/ mouse) on the morning of the seventh day, while the other half received the normal progesterone injection. Mice were sacrificed 24 hours later for analysis by in situ hybridization.

The effect of steroids on uterine development in the absence of blastocysts was examined by ovariectomizing mice and treating them with hormone injections after a two week recovery period. They received injections of either progesterone (1 mg/ mouse), estrogen (100 ng/ mouse), or a combination of both (10 ng estrogen and 1 mg progesterone/mouse) for three days. Mice were then sacrificed on the morning of the fourth day. To further determine the importance of progesterone on ISP2 gene expression superovulated pregnant and pseudo-pregnant mice (Hogan et al., 1994) were treated with RU486 (400 μg/mouse) on the morning of the third day after a vaginal plug was detected. The mice were then sacrificed 24 hours later and their uterine horns were collected for in situ hybridization analysis.

Embryo Culture

Morulae were collected from oviducts of superovulated, 2.5 day pregnant dams in M2 medium (Hogan et al., 1994). For hatching, morulae were cultured in microwells for approximately 24 hrs at 37° C., 5% CO2 in KSOMaa medium (Erbach et al., 1994). In embryo outgrowth, hatched blastocysts were cultured for an additional 48 hrs at 37° C., 5% CO2 in Dulbecco's Modified Eagle's Medium plus 5% (v/v) fetal bovine serum on microwells coated with extracellular matrix derived from 10% (v/v) Triton X-100-treated mouse embryo fibroblasts (Behrendtsen et al., 1995).

Embryo RNA Preparation

Total RNA was collected from embryos plus deciduum (E6.5 implantation sites), embryos (E8.5, 11.5, E13.5) and placentas (E11.5, E13.5) using Trizol (Life Technologies). Hatching blastocysts (at 50% hatch) were collected by centrifugation for Trizol RNA preparation. RNA from outgrowing blastocysts was collected by Trizol lysis directly in microwells. Poly (A)⁺ RNA was enriched from E6.5 embryo/deciduum total RNA using oligo (dT) cellulose chromatography (Sambrook et al., 1989).

Serine Proteinase Active Site RT-PCR Cloning

Total RNA (1 μg) from E6.5 embryo/deciduum was reverse transcribed using Superscript II (Life Technologies) and used as a template for active site PCR using degenerate His (5′-CGGAATTCTI(ACT)TI(AT)(GC)IGC(AGCT)G(AGCT)CA(CT)TG-3′; SEQ ID NO:7) and Ser

(5′-GCGGATCCA(AG)IGGICCICC(ACGT)(CG)(TA)(AG)TC(AGCT)CC-3′; SEQ ID NO:8) active site primers (Prendergast et al., 1991). Each 12.5 μl PCR reaction utilized 0.5 μl of cDNA in 10 mM Tris-HCl, pH 9.0, 50 mM KCl, 1.5 mM MgCl2, 130 μM dNTPs, 1 μM of each primer and 1U Taq polymerase (Amersham Pharmacia). Forty rounds of thermal cycling consisted of 1 min at 94° C., 2 min at 55° C., and 2 min at 72° C. The amplification products were ethanol precipitated, cleaved at flanking 5′ EcoRI and 3′ BamHI sites designed in the primer ends, were eluted from a 1% (w/v) agarose gel and cloned into EcoRI/BamHI cut pBluescript KS⁺ (Stratagene). The inserts of individual clones were screened by restriction analysis (Sambrook et al., 1989), dye-terminator sequenced (PE Biosystems) and compared to the Genbank sequence database using the BLAST program provided by the NCBI network server (Altschul et al., 1997).

Library Construction. cDNA Cloning and Sequence Analysis

10 μg of poly(A)⁺ RNA (E6.5 embryo/deciduum) was converted to random- and oligo(dT)-primed double stranded cDNA using the SuperScript cDNA cloning kit (Life Technologies). NotI - EcoRI adapters were ligated to the cDNA; the adapted cDNA was size selected by gel exclusion chromatography using a Sephacryl S-500 HR column (Life Technologies) and excess linkers were removed using Gene Clean (Bio 101). 1 μg of adapted cDNA was ligated with 5 μg of dephosphorylated, EcoRI-cut pGT10 (Amersham Pharmacia) and packaged into phage using Gigapack Gold II (Stratagene). 2×10⁶ recombinant phage were amplified on plates and the pooled lysates were frozen (−80° C.) in 7% (v/v) DMSO (Sambrook et al., 1989).

A 478 bp ISP1 PCR sub-fragment, or a 478 bp ISP2 PCR sub-fragment, were used to screen 5×10⁵ plaques from this library and resulted in the identification of, for each of the probes used, two cDNA clones bearing a 1.3 kb insertion. An internal BamHI site within the ISP1 cDNA clone permitted the directional cloning of 0.5 and 0.8 kb EcoRI-BamHI fragments into pBluescript KS⁺ (Stratagene) for cycle sequencing (PE Biosystems). The nucleotide sequence was translated into protein sequence using the Swiss Protein ExPAsy tool (http://expasy.cbr.nrc.ca/tools/dna.html). Twelve serine proteinase peptides identified from a BLAST identity search were aligned with ISP1 using Clustal W (Higgins, 1994; http://dot.imgen.bcm.tmc.edu:9331/multi-align/multi-align.html).

For ISP2, The 1.3 kb Eco RI fragment was subcloned into pBKCMV (Stratagene) for cycle sequencing (PE Biosystems). The nucleotide sequence was translated into protein sequence using the Swiss Protein ExPAsy tool (http://expasy.cbr.nrc.ca/tools/dna.html). Nine serine proteinase peptides identified from a BLAST identity search were aligned with ISP2 using Clustal W (Higgins et al., 1994; http://dot.imgen.bcm.tmc.edu: 9331/multi-align/multi-align.html) and compared to develop a dendrogram using the protein parsimony method (http:/Ibioweb.pasteur.fr/seqanal/phylogeny/ phylip-uk.html).

Expression Analysis

5 μg of poly (A)⁺ RNA from E6.5 embryo/deciduum was electrophoresed through a 1.2% (w/v) formaldehyde-agarose gel alongside an RNA high molecular weight ladder (Life Technologies). After transfer to Hybond N⁺ (Amersham Pharmacia), the membrane was probed with the 1.2 kb, ³²p -labeled ISP1 cDNA fragment, or with the 1.2 kb, 32P-labeled ISP2 cDNA clone.

The presence of ISP1 transcripts in embryos and placentas was monitored using RT-PCR. Total RNA (1 μg) was reverse transcribed and amplified using ISP1 specific primers (ISP1 for 5′-GGAGCAGGAACTTCTGAACA-3′; SEQ ID NO:9 and ISP1 rev 5′-GTCAAAGATGGCCACAGC-3′: SEQ ID NO:10) and forty rounds of thermal cycling (1 min at 94° C., 2 min at 60° C., and 2 min at 72° C.). The RT-PCR amplification of GAPDH (as a control for mRNA loading) is described elsewhere (Arcellana-Panlilio and Schultz, 1993). The predicted 175 and 380 bp amplification products were separated on a 2% (w/v) agarose gel.

The presence of ISP2 transcripts in embryos and placentas was monitored using the same methods and ISP2 specific primers, (ISP2for:

5′-TGTGAGCCGGGTCATCATCC-3′; SEQ ID NO:11 and ISP2rev

:5′-GGCATTGTGGTACATCTCCT-3′; SEQ ID NO:12). The predicted 175 and 360 bp amplification products were separated on a 2% (w/v) agarose gel.

Whole embryo RNA in situ hybridization using digoxigenin-labelled RNA probes was performed essentially as previously described (Rancourt and Rancourt, 1997). The ISP1 probe comprised the 478 bp RT-PCR subclone in pBSKS⁺. The ISP2 probe also comprised a 478 by RT-PCR subclone in pBSKS⁺. The antisense probes were synthesized using T3 polymerase after plasmid linearization with EcoRI. The sense probes were synthesized using T7 polymerase after plasmid linearization with BamHI. All experiments were performed with the sense RNA probe in parallel to detect non-specific hybridization. Histochemical staining of strypsin activity was performed essentially as outlined previously (Perona and Wassarman, 1986). Embryos were collected as early blastocysts in M2 medium, and were lightly fixed in 1.25% (w/v) glutaraldehyde in 0.25M sucrose, 50 mM sodium phosphate (pH 7.5) for five min at 4° C. Following fixation, the blastocysts were placed in 50 mM sodium phosphate (pH 7.5) containing the substrate N-α-benzoyl-DL-arginine B-napthylamide (0.56 mM; Sigma) and Fast Garnet GBC salt (1.86 mM; Sigma), were incubated for 5 min at room temperature and washed in 50 mM sodium phosphate (pH 7.5).

Antisense Oligodeoxynucleotide Studies

In antisense oligodeoxynucleotide studies, harvested blastocysts were placed in 0.001% (v/v) L-a-lysophosphatidylcholine for 60 seconds (Jones et al., 1997) and were transferred to microdrops equilibrated with 2.5 μM, 5 μM oligodeoxynucleotide or an equal volume of H20 (Behrendtsen et al., 1995). Two antisense oligodeoxynucleotides were designed against regions surrounding ISP1's initiation codon: AS1

(5′-TCTAACTACCGTCTAACAACG-3′; SEQ ID NO;13) situated upstream, AS2 (5′-GAACTCTTCTAACTACCGTCT-3′; SEQ ID NO:14) lying downstream. A control oligodeoxynucleotide, SS1 (5′-ACGGTAGTTAGAAGAGTTCT-3′; SEQ ID NO:15), represented the scrambled sense sequence surrounding the initiation codon. The oligodeoxynucleotides were designed using OligoTM software and were synthesized and purified by Dr. Richard Pon, UC DNA Services (University of Calgary). Blastocysts were scored at 20, 30, 40, and 60 hours for progress in hatching. In these studies, both AS 1 and AS2 interfered specifically with blastocyst hatching. However AS1 was found to be more effective than AS2 and was used in all subsequent experiments. Following eight hours of treatment, some blastocysts were assayed for the presence of ISP1 transcripts using RT-PCR. Following 24 hours of treatment, some blastocysts were assayed for strypsin activity using histochemical staining. In outgrowth studies, blastocysts were allowed to hatch and then transferred to microdrops equilibrated with oligodeoxynucleotide or water.

Progress in outgrowth was monitored over a period of 5 days.

Recombinant Protein Production

(a) Small Scale Protein Production: DNA fragments from ISP1 and ISP2 were amplified by PCR, using the following primers:

| | |
|---|---|
| i) For ISP1- | |
| 5′-GCGGATCCGTGGGGGAAGTA-3′ and | (SEQ ID NO:16) |
| 5′GCGAATTCAGCTTTGTGCTCGTC-3′ | (SEQ ID NO:17) |
| ii) For ISP2- | |
| 5′ GCGATCCTATGGGGGCAAG-3′ and | (SEQ ID NO:18) |
| 5′GCGAATCGTGGTACATCTC-3′. | (SEQ ID NO:19) |

The PCR fragments were ligated into the BamH1 and EcoR1 sites of pGEX-2T (Pharmacia), transformed into the E. coli strain BL21 and plated on NZY-Ampicillin plates. A single colony of transformed bacteria was used to inoculate 2.5 ml of YT(2X)-Ampicillin (100 mg/ml) medium and grown overnight at 37° C. 100 ml of this culture was used to inoculate 5 ml of YT-Ampicillin and shaken at 37° C. for 5 hours, until an OD600 of 0.5 was reached. 10 ml of 100 mM IPTG was added to induce GST fusion protein expression and the culture was incubated at 30° C. 3-5 hrs. 1.5 ml of this culture was centrifuged at 13,000×g to pellet the cells, the supernatant was discarded. The pellet was washed once with 200 ml ice-cold STE and then resuspended in 300 ml ice-cold STE. This was vortexed for 5 seconds and sonicated on ice for 10 seconds until the liquid was clear. Following centrifugation for 5 min at 4° C. the supernatant was transferred to a new tube. Triton X 100 was added to a final concentration of 20%, and after vortexing the tube was rocked for 30 min at 4° C. The extract was centrifuged at 13,000×g for 5 min to remove insoluble debris, the supernatant was transferred to a new tube and the pellet resuspended in 300 ml of ice cold STE. 10 ml aliquots of both the supernatant and suspended pellet were resolved on 10% SDS PAGE gel.

(b) Large Scale Production. 2 ml of overnight culture was used to inoculate 0.5L of YT(2X)-Ampicillin (100 mg/ml) and grown to an OD600 of 0.5-1.0 at 37° C. IPTG was added to a final concentration of 0.5 mM, to induce expression of the fusion protein, and the culture was incubated at 30° C. overnight with shaking. The bacteria were pelleted by centrifugation at 7,000×g for 5 min, and resuspended in STE to a final concentration of 10% vol/vol. Lysozyme was added, to a final concentration of 1 mg/ml and the cells were incubated at room temperature for 20 min. The mixture was centrifuged at 5000×g for 10 min, the supernatant discarded and the pellet kept on ice until it was resuspended in 10 ml ice-cold STE containing 0.1% sodium deoxycholate. This solution was incubated on ice with occasional mixing for 10 min, then MgCl, to final concentration of 8 mM and DNAse I, to a final concentration of 10 mg/ml, were added. The solution was incubated at 4° C. with occasional mixing until the viscosity disappeared, and the inclusion bodies were removed by centrifugation. The pellet was washed once by resuspension in STE containing 1% NP-40 and centrifugation, and washed again by resuspension with STE and centrifugation. The pellet was resuspended in 2.5 ml STE and sonicated three times for 30 seconds each time. 6×loading buffer was added, the solution was boiled, and the proteins were resolved in 10% SDS PAGE gel.

Antibody Production

Antibodies to the gel purified GST fusion proteins were made in rabbits. Total protein was separated out on a 10% SDS PAGE gel and stained for 10 min in 0.05% Coomassie blue in water, and subsequently destained in water. The fusion protein was identified and excised from the gel. The gel slice was frozen in liquid nitrogen and ground into a powder with mortar and pestle. The powder was resuspended in DPBS and approximately 100 μg of the fusion protein was mixed with an equal volume of Freund's adjuvant (DIFCO) and injected subcutaneously into New Zealand white rabbits. Three weeks later a sample of blood was collected from the rabbits and they were boosted with 100 μg of fusion protein in incomplete Freund's adjuvant (DIFCO). Subsequent boosts/bleeds followed every three weeks.

### Example 1

### ISP1 Represents a Novel Branch of the Tryptase Subfamily of S1 Proteinases

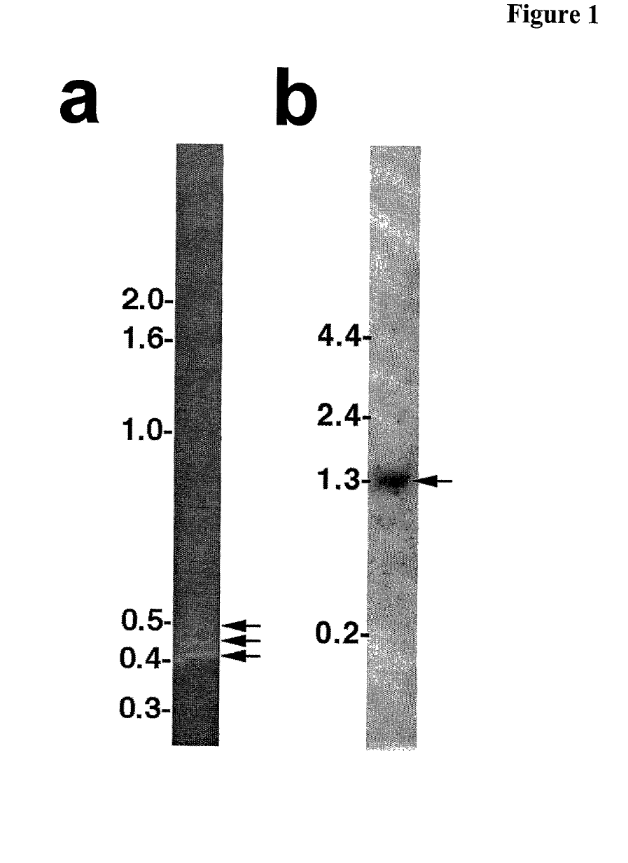

Previous investigations of implantation, both in vivo and in vitro via blastocyst invasion assays, have indicated that a cascade of proteinases mediates the embryo/uterine interaction and the integration of the embryo into uterine deciduum during implantation. Active site RT-PCR (Prendergast et al., 1991) using RNA from day 6.5 implantation sites and embryos was used to identify novel serine proteinase sub-cDNAs that were expressed at around the time of implantation using degenerate primers. At this stage in implantation the embryo is fully engaged in invading the deciduum. Amplification with degenerate primers surrounding the active site His and Ser regions gave rise to a number of fragments ranging in size between 0.4-0.5 kb, consistent with the size of known serine proteinase His-Ser sub-cDNAs (FIG. 1a). Upon cloning and sequence characterization of these sub-cDNAs, a number of serine proteinases were identified including urokinase plasminogen activator, tissue plasminogen activator, granzyme D, granzyme F and two previously unidentified genes which are herein referred to as Implantation Serine Proteinases (ISPs), and more specifically, ISP1 and ISP2.

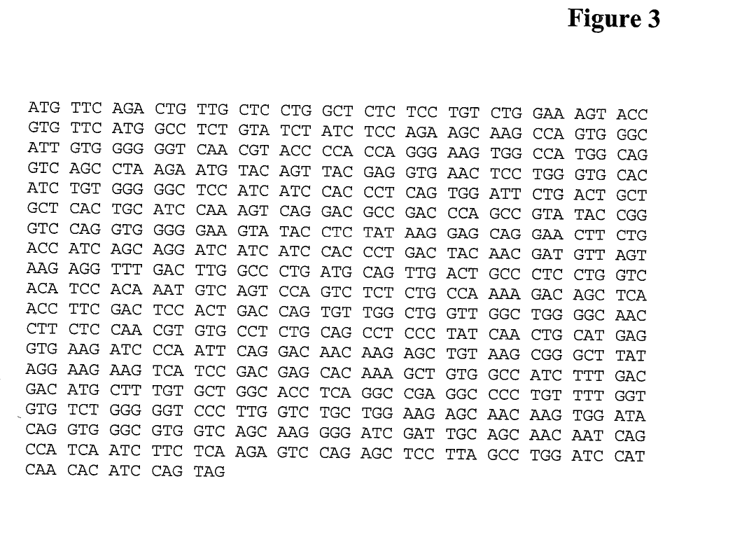

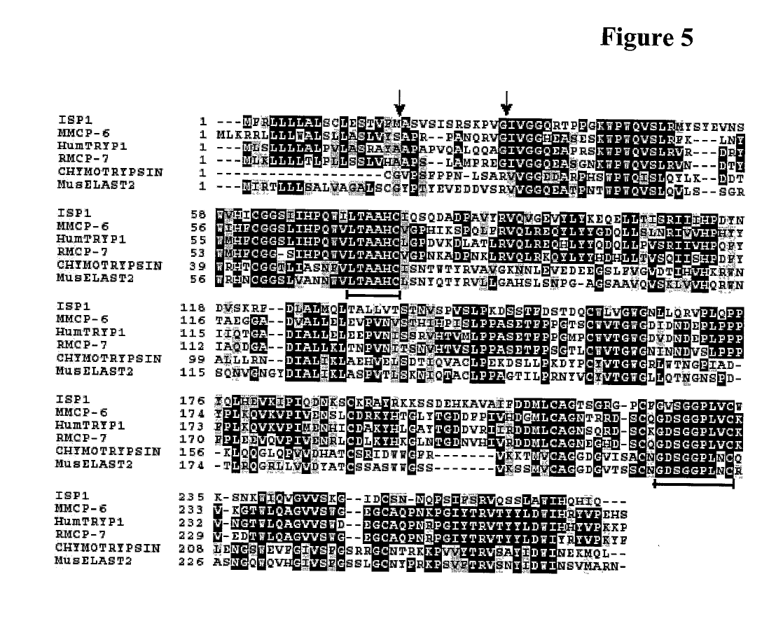

Northern analysis of 6.5 day embryo/deciduum poly (A)⁺ RNA using the 478 bp ISP1 sub-cDNA fragment indicated that the ISP1 transcript was 1.3 kb (FIG. 1b). A 1.2 kb full-length eDNA clone for ISP1 was isolated by generating and screening a 6.5 day mouse embryo/deciduum cDNA library in 1GT10. Sequence analysis of ISP1 (FIG. 3) revealed a predicted protein of 273 amino acids in length (FIG. 5). In BLAST identify searches (Altschul et al., 1997) ISP1 was found to share high degrees of sequence similarity with the haematopoietic serine proteinases, the most similar being Mouse Mast Cell Protease 6 (45% amino acid identity; Lutzelschwab et al., 1997). Other mast cell proteinases showed similar degrees of sequence identity. The next closest subfamilies contain chymotrypsins and elastases, with approximately 38% identity. The relationship of ISP1 to the S1 peptidase family is clear as it shares the conserved His and Ser active site moieties (LTAAHC and GDSGGPL), in addition to the common N-terminal sequence (IVGG; SEQ ID NO:24) of mature tryptases (FIG. 5; Smyth et al., 1996).

### Example 2

### ISP2 Represents Another Novel Branch of the Tryptase Subfamily of S1 Proteinases

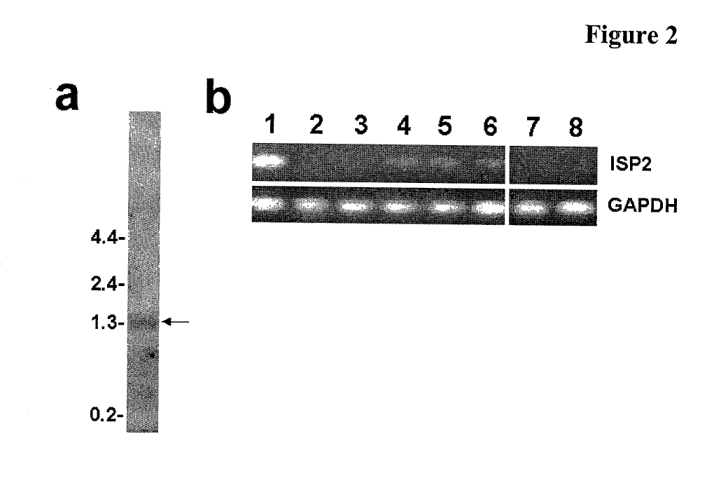

Using a 478 bp ISP2 cDNA fragment derived from active site RT-PCR, a 6.5 day mouse embryo/deciduum CDNA library was screened and a 1.2 kb cDNA clone was identified. Northern analysis of 6.5 day embryo/deciduum poly (A)⁺, which revealed a single 1.3 kb mRNA species when hybridized with the 1.2 kb ISP2 cDNA clone (FIG. 2a), suggesting that, as with ISP1, this cDNA was full length.

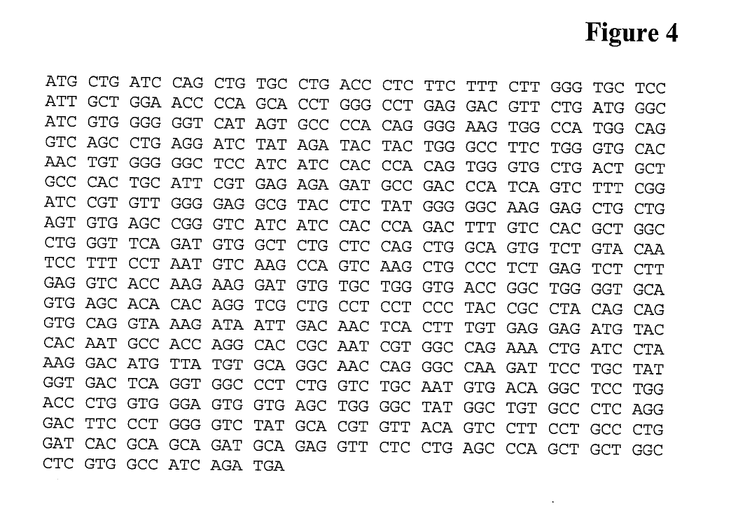

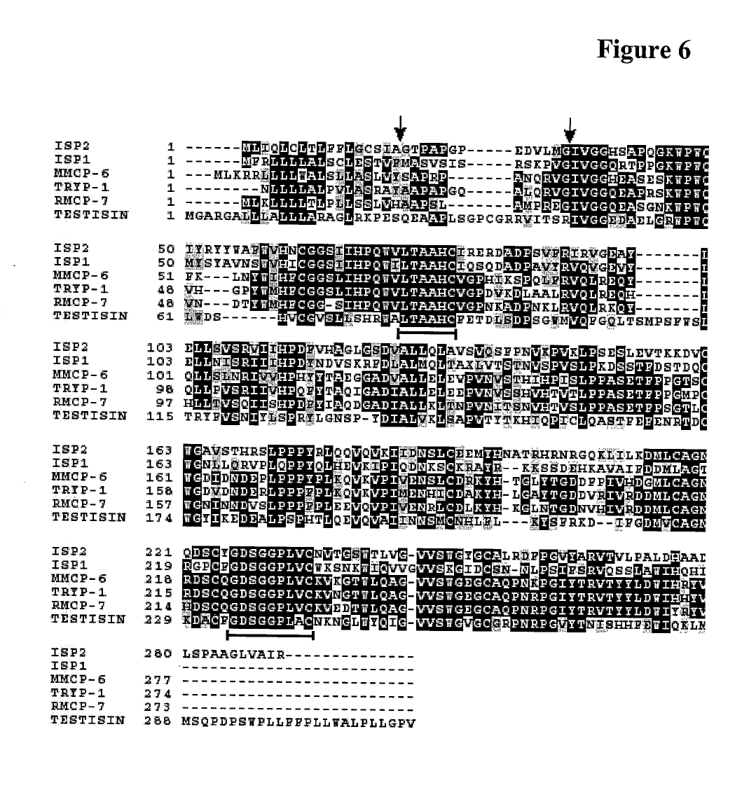

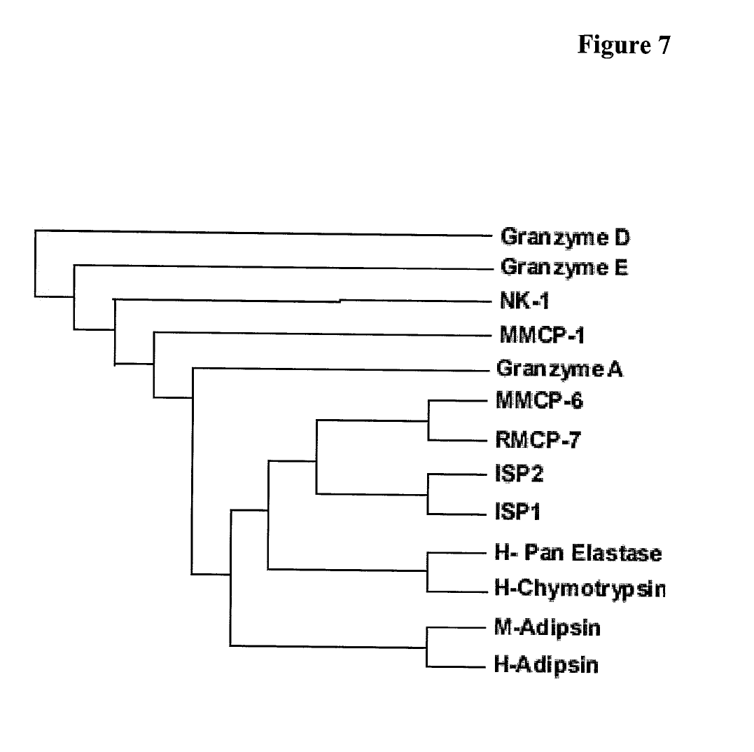

Sequence analysis of ISP2 (FIG. 4) revealed a predicted protein of 290 amino acids in length (FIG. 6). BLAST identity searches (FIG. 2; Altschul et al., 1997), revealed that ISP2 shared a moderate amount of sequence similarity with ISP1 and hematopoietic tryptases (MMCP6, 45% amino acid identity; Lutzelschwab et al., 1997). Other mast cell proteinases showed similar degrees of sequence identity. The next closest subfamilies contain chymotrypsins and elastases with approximately 34% identity. Like ISP1, the relationship of ISP2 to the S1 peptidase family is clear, as it shares the conserved His and Ser active site moieties (LTAAHC and GDSGGPL, respectively), in addition to the common N-terminal sequence (IVGG) of mature tryptases (FIG. 6c; Smyth et al., 1996). Maximum parsimony analysis (Higgins et al., 1994) suggests that based on the low degree of similarity between the ISPs, and their nearest neighbors within the mast cell tryptase family, the ISPs represent a distinct branch of the S1 proteinase superfamily that diverged from the elastase/chymotrypsin and mast cell proteinase clusters at approximately the same time (FIG. 7).

### Example 3

### ISP1 Gene Expression in Preimplantation Embryos and In Vitro Hatching and Outgrowth

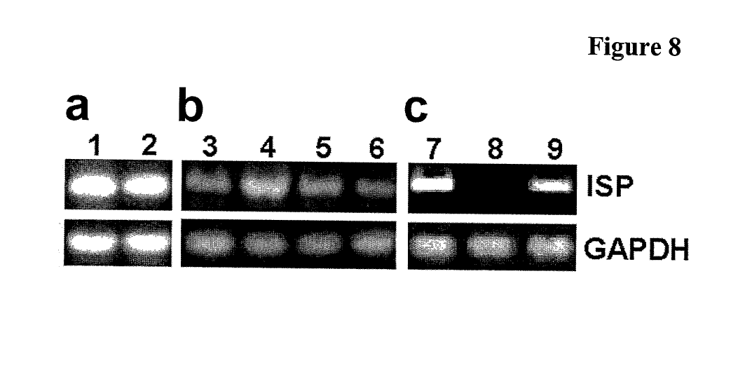

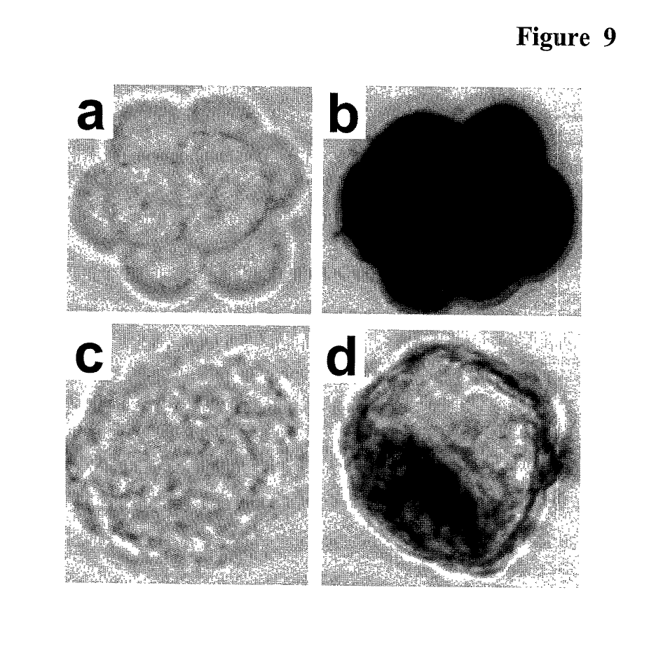

Given its sequence properties, it was hypothesized that the ISP1 gene encodes the previously described trypsin-like proteinase, strypsin, involved in blastocyst hatching (Perona and Wassarman, 1986). Consistent with this hypothesis, RT-PCR confirmed that ISP1 is expressed during hatching and embryo outgrowth (FIG. 8a), and is detectable throughout all stages of pre-implantation development, as early as the zygote stage (FIG. 3b). Beyond implantation, ISP1 expression was detected faintly in day 11.5 and 13.5 placenta, but not in day 8.5, 11.5, or 13.5 embryos. In agreement with previous RT-PCR expression data, stronger in situ hybridization staining was observed in morulae compared to blastocysts (FIG. 9). In the blastocyst, ISP1 RNA expression was observed throughout the embryo. Here, equivalent staining of blastomeres was noted, although staining appeared stronger within the multilayer inner cell mass, than in the monolayer trophoblast (FIG. 9d).

### Example 4

### Antisense Abrogation of ISP1 Gene Expression and Strypsin Activity in Blastocysts

A possible role for ISP1 in hatching was examined by determining whether ISP1 mRNA could be reduced in blastocysts by treatment with antisense oligodeoxynucleotides (Behrendtsen et al., 1995; Jones et al., 1997) and result in an alteration of the hatching process. Two antisense oligodeoxynucleotides were designed surrounding ISP1's initiation codon covering the region immediately upstream (AS1) and downstream (AS2). A control oligodeoxynucleotide (SS1) represented the scrambled sense sequence surrounding the initiation codon. Blastocysts were scored at 20, 30, 40, and 60 hours for progress in hatching. In these studies, both AS1 and AS2 interfered specifically with blastocyst hatching (Table 1). AS1 was more effective than AS2, and was used in all subsequent experiments.

TABLE 1
Unhatched Mouse Blastocysts after Oligodeoxynucleotide Administration

| | Percent Hatched over Time (100 blastocysts) | | | |
|---|---|---|---|---|
| Oligodeoxynucleotide | 20 hours | 30 hours | 40 hours | 60 hours |
| Blank | 10 | 30 | 65 | 80 |
| SS1 (2.5 μM) | 10 | 16 | 58 | 90 |
| AS1 (2.5 μM) | 0 | 0 | 16 | 37 |
| AS2 (2.5 μM) | 0 | 20 | 35 | 40 |
| SS1 (5.0 μM) | 10 | 39 | 61 | 77 |
| AS1 (5.0 μM) | 10 | 10 | 20 | 20 |
| AS2 (5.0 μM) | 0 | 5 | 35 | 45 |

SS1 is a scrambled sense oligodeoxynucleotide of DNA sequence surrounding the initiation codon of ISP1. AS1 and AS2 are antisense oligodeoxynucleotides surrounding ISP1's initiation codon covering the region immediately upstream (AS1) and downstream (AS2).

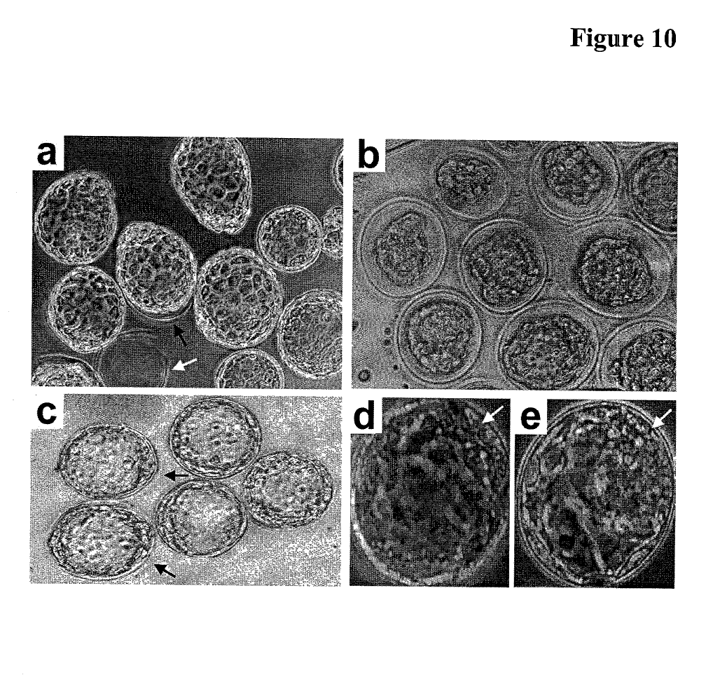

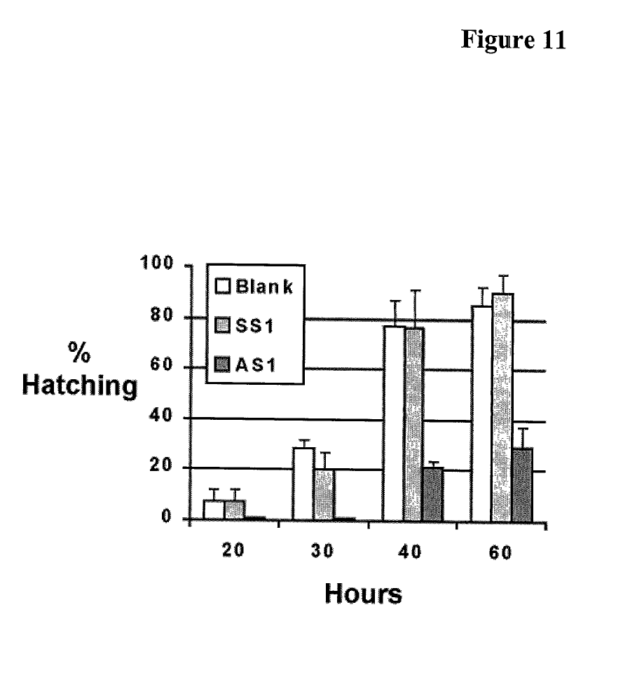

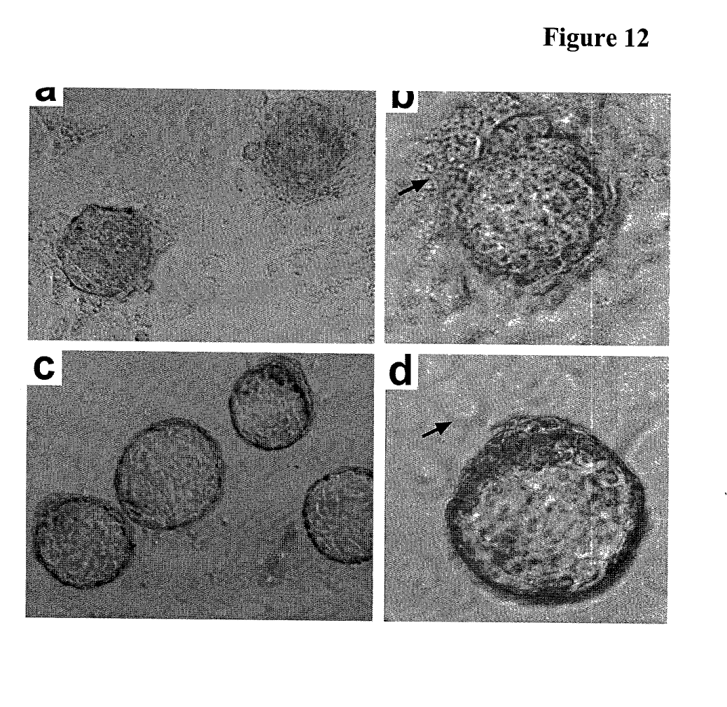

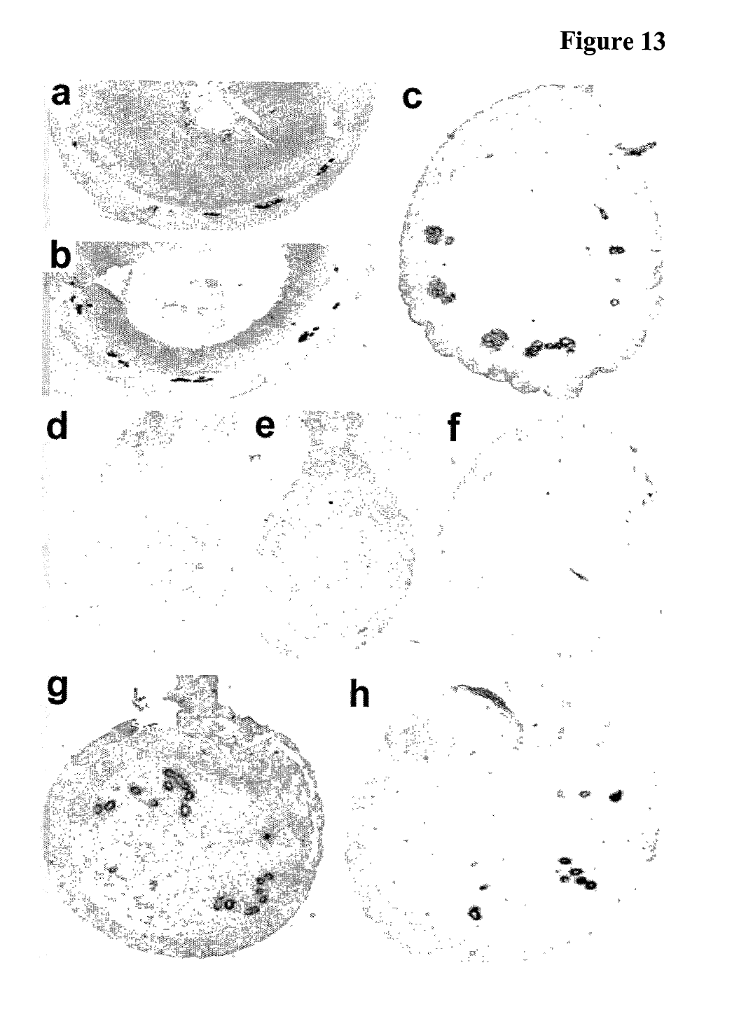

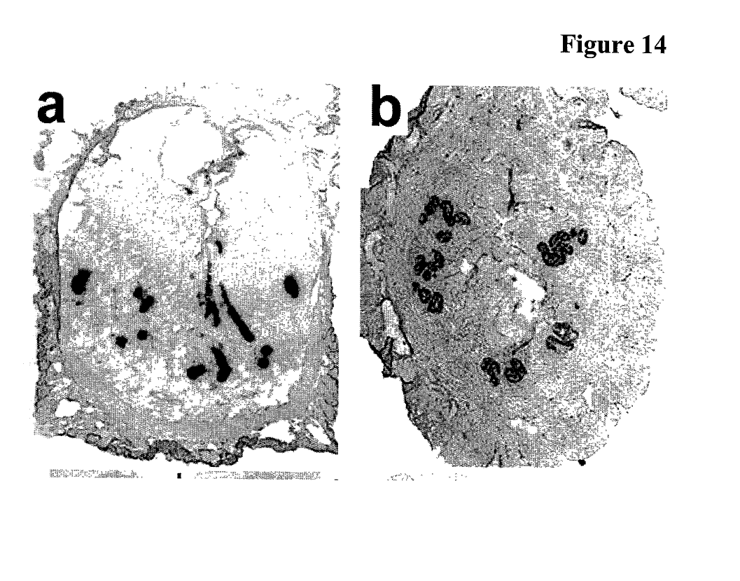

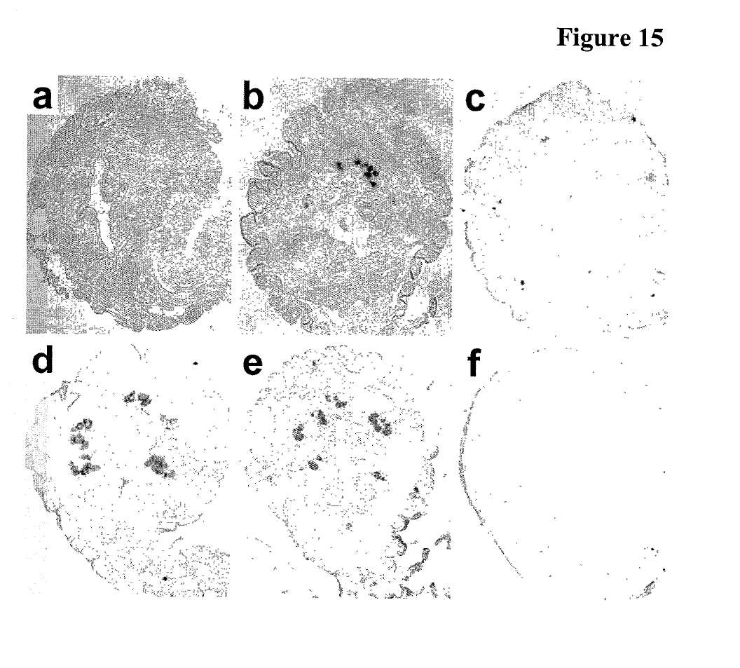

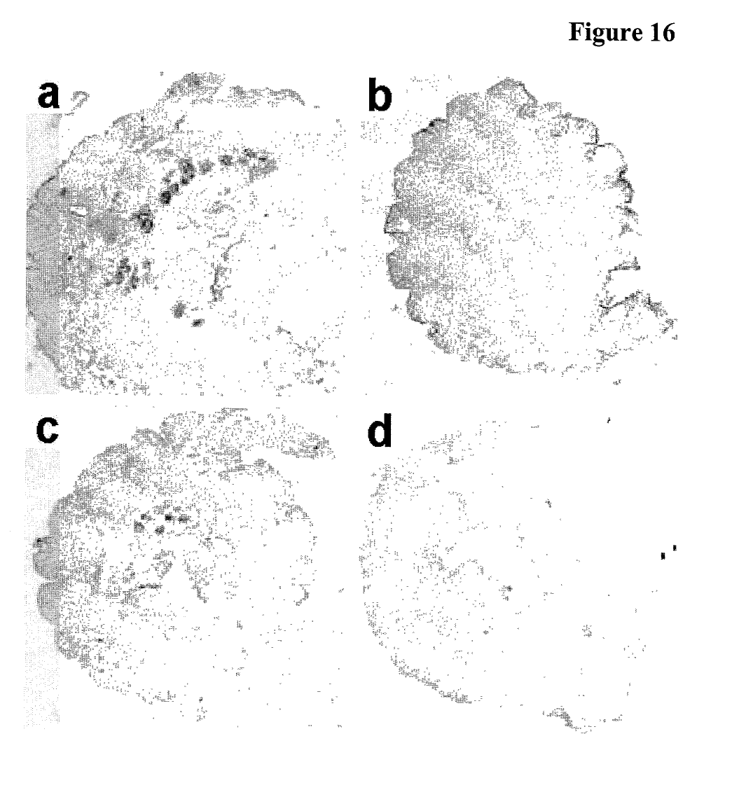

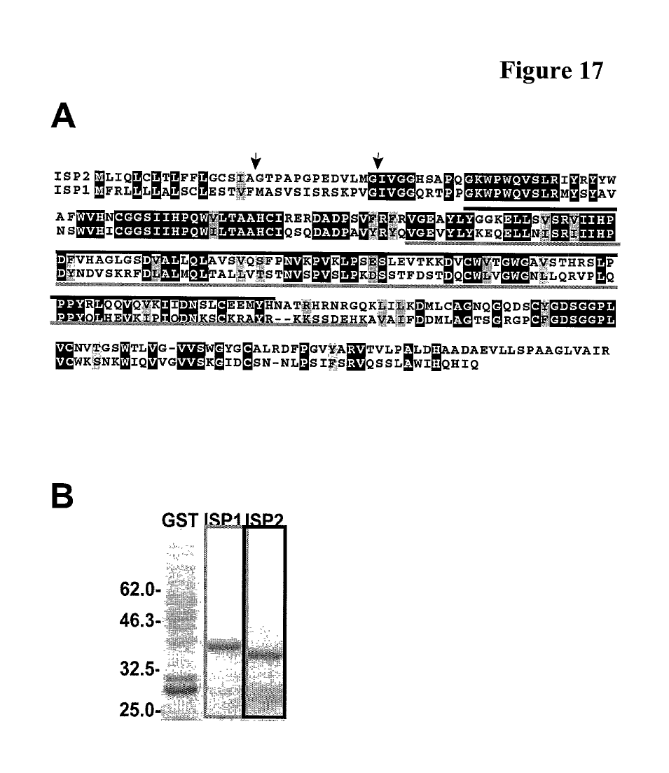

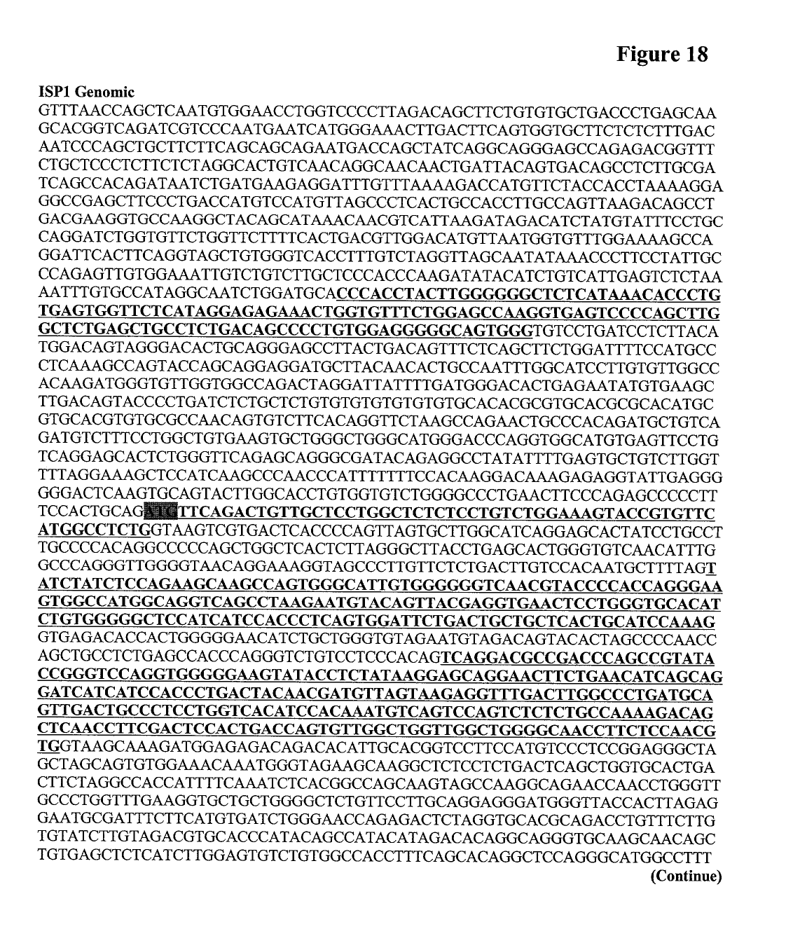

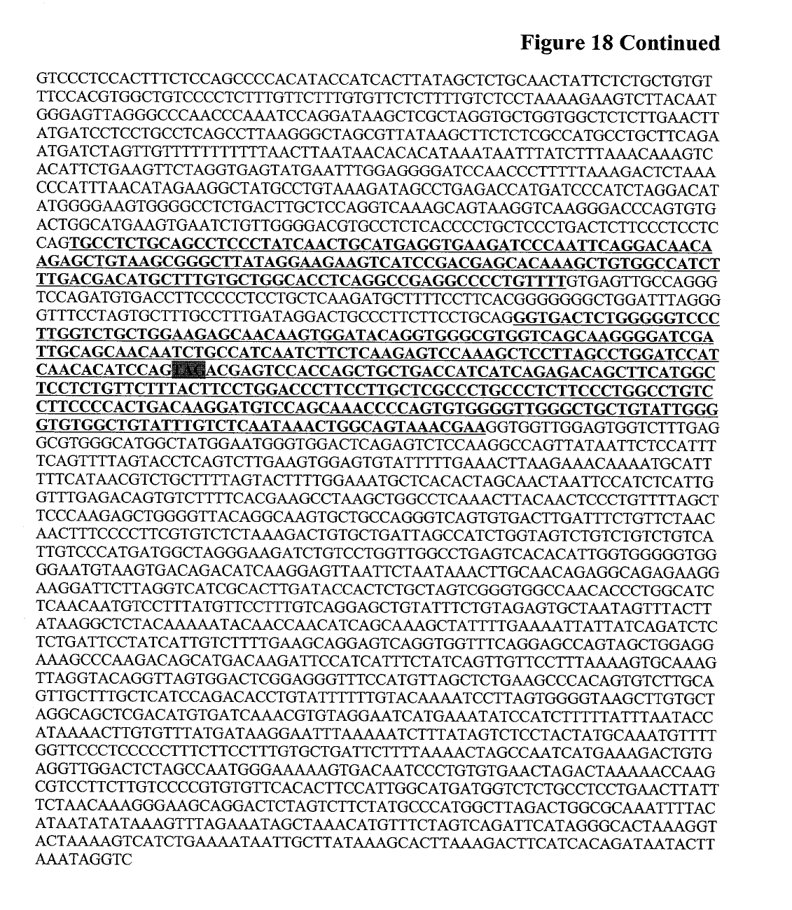

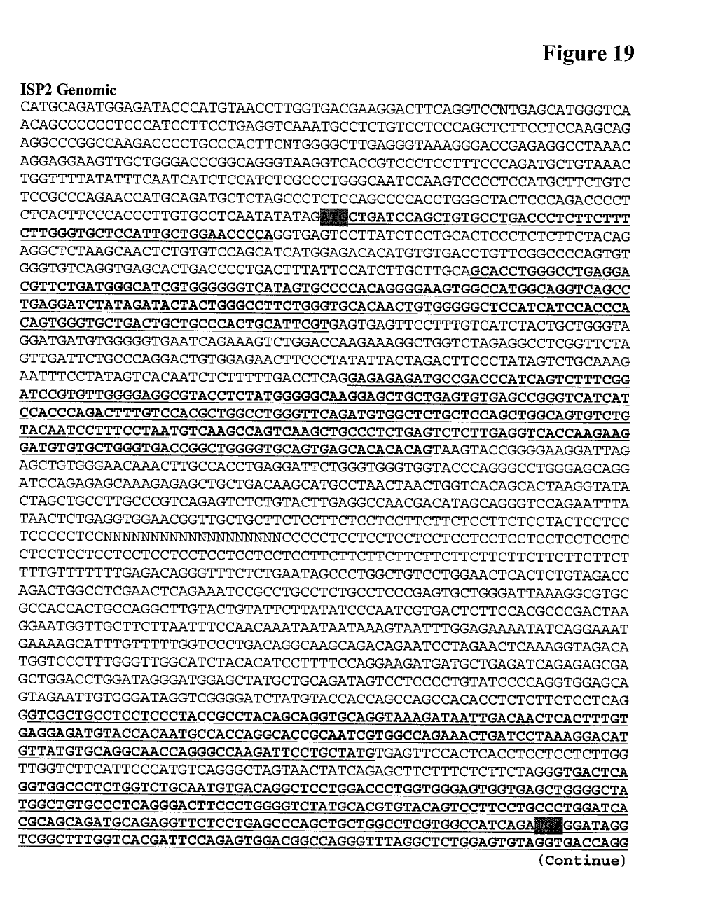

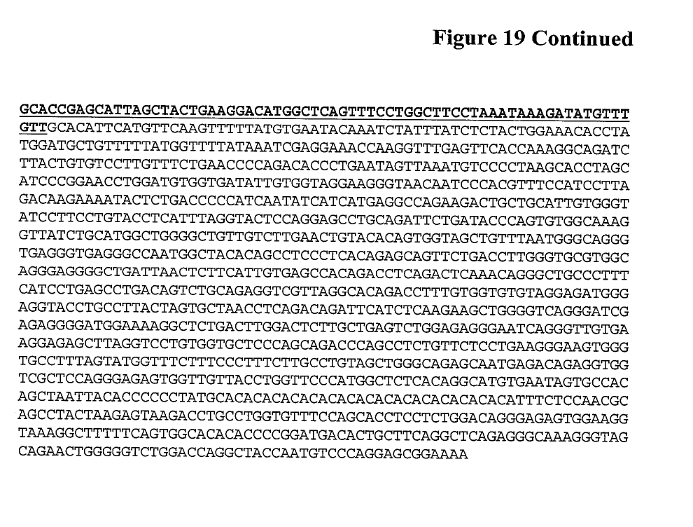

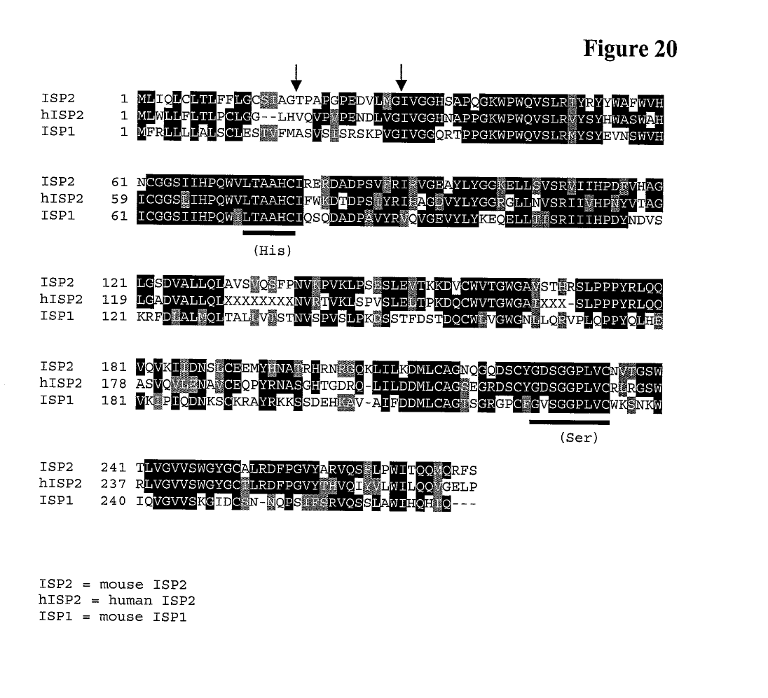

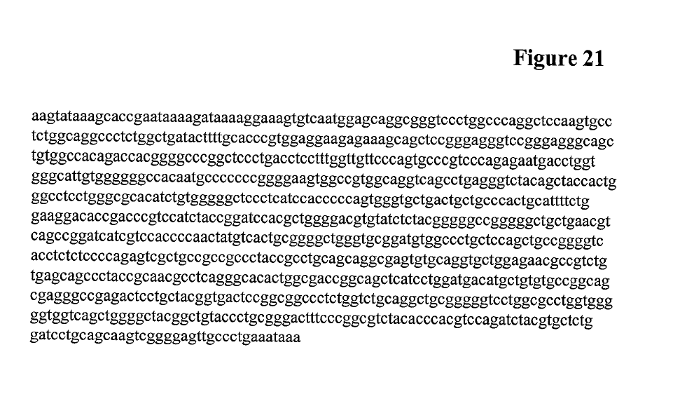

When blastocysts were treated with the AS1 oligodeoxynucleotide, the accumulation of ISP1 transcripts was reduced at least 100-fold after eight hours of culture (FIG. 8c). A significant reduction was not observed in the corresponding SS1 control oligodeoxynucleotide-treated blastocysts, which had similar ISP1 transcript levels as untreated controls. AS 1 oligodeoxynucleotide-treated blastocysts also displayed reduced strypsin activity compared to untreated or control oligodeoxynucleotide-treated blastocysts. In control-oligonucleotide treated blastocysts (FIG. 10d), localized strypsin activity was observed histochemically at the abembryonic pole of blastocysts. In contrast, strypsin activity was absent in antisense oligodeoxynucleotide-treated blastocysts (FIG. 10e). Consistent with our hypothesis, this observation suggested that the ISP1 gene encodes the strypsin activity that is responsible for hatching.

### Example 5

### Disruption of Hatching In Vitro with ISP1 Antisense RNA

Blastocysts treated with the antisense oligodeoxynucleotides displayed a considerable impairment in hatching over time (Table 1). Compared with control oligodeoxynucleotide-treated and untreated controls, a significant percentage of antisense oligodeoxynucleotide-treated blastocysts did not hatch (FIG. 11). Others displayed a delay in hatching, suggesting that antisense treatment could transiently inhibit hatching until the concentration of oligodeoxynucleotide in the media declined.

Control oligodeoxynucleotide-treated blastocysts developed and hatched normally (FIG. 10a), mirroring untreated blastocysts that were cultured in parallel (not shown). Beginning around 20 hours, the zona became thin and the blastocysts emanated through ruptures at the abembryonic pole. In contrast, the majority of antisense oligodeoxynucleotide-treated blastocysts grew until they compressed and thinned the zona wall (FIG. 10c), but were unable to cause it to rupture. After 60 hours inside the zona, antisense treated blastocysts began to die and shrink away from the wall (FIG. 10b).

### Example 6

### Disruption of Blastocyst Invasion with ISP1 Antisense RNA

Since ISP1 is expressed during blastocyst outgrowth in vitro, the effect of antisense RNA on blastocyst outgrowth after hatching was studied (FIG. 6). Control oligodeoxynucleotide-treated blastocysts adhered to and invaded extracellular matrix after 2 days in culture, not unlike untreated control blastocysts (FIG. 12 a, b). ISP1 antisense oligodeoxynucleotide-treated blastocysts took 5 days to adhere to the matrix, but growth was so delayed that the blastocysts never reached the size or extent of the outgrowth observed with control embryos (FIG. 12 c, d). This observation suggested that ISP1 expression is also vital for the initiation of blastocyst attachment to extracellular matrix and subsequent outgrowth during implantation.

### Example 7

### Temporal Expression of ISP2 During Gestation

RT-PCR was use to characterize the expression of ISP2 throughout gestation (FIG. 2b). Strong expression was observed in E6.5 embryo/deciduum RNA consistent with the expression observed using northern blotting. Weaker expression was also observed in placental RNA isolated from E11.5 and E13.5 pregnancies. ISP2 gene expression was not observed in RNA from the embryo proper at 8.5 and 11.5 days; a residual amount of expression was detected at 13.5 days. This pattern of expression for ISP2 resembled that previously identified for ISP1. Based on ISP1's role in blastocyst hatching and outgrowth, it was of interest to investigate whether ISP2 might also be expressed in the blastocyst and have a similar role to ISP1. However, RT-PCR of RNA isolated from blastocysts hatched or outgrown in vitro indicated that ISP2 was not expressed in the early embryo (FIG. 2b). This result was confirmed by additional in situ hybridization experiments performed on morula and blastocysts, which indicated that ISP2 is not expressed (data not shown). Based on these expression results, it appeared that ISP2's function was distinct from that of ISP1 and that ISP2's function likely resided within the uterine deciduum during implantation.

### Example 8

### ISP2 is Expressed in Glandular Epithelium during Implantation

In situ hybridization analysis of sagittal sections confirmed that ISP2 gene expression originated from the deciduum. Throughout the peri-implantation period, strong ISP2 mRNA staining was observed specifically within endometrial gland epithelium (FIG. 13). Expression was identified first in sagittal sections of E6.5 implantation sites (not shown) and subsequently in implantation sites of E7.5 and E8.5 pregnancies (FIG. 13a, b). At day 6.5, ISP2 mRNA staining was also observed between implantation sites lying remote from the embryo (FIG. 13c). These results suggested to the Applicants that ISP2 gene expression might not be restricted to or dependant solely upon decidua surrounding the implantation site. However, ISP2 mRNA staining was not observed in virgin uterus (FIG. 13d), which suggested that ISP2 gene expression occurred specifically in response to pregnancy. ISP2 gene expression was also not observed on day 2.5 (FIG. 13e), when the morula is in the oviduct, or on day 3.5 (FIG. 13f), when the blastocyst enters the uterus. However ISP2 niRNA staining was observed on day 4.5 and day 5.5 (FIG. 13g, h), when the implantation window is opened. These results suggested that ISP2 expression occurs either in response to the implantation reaction or is hormonally regulated in synchrony with implantation.

### Example 9

### ISP2 Gene Expression in Pseudo-Pregnancy

The potential role of hormones and the decidualization reaction in regulating ISP2 gene expression was investigated by inducing artificial pregnancies in ovariectomized females using uterine oil injections after progesterone and estrogen priming (Finn, 1966; Milligan, 1995). As part of the experimental design, oil was introduced into only one uterine horn to ensure that the decidualization reaction occurred only on one side of the animal. The other side served to control for the potential role of hormonal treatments on ISP2 gene expression. Following in situ hybridization, ISP2 mRNA staining was observed in both uterine horns, suggesting that ISP2 gene expression also occurs during artificial pregnancy and in the absence of decidualization (FIG. 14).

### Example 10

### ISP2 Gene Expression is Induced by Progesterone

The influence of steroid hormones was examined using models of pregnancy and pseudo-pregnancy. In delayed implantation experiments, ISP2 gene expression was abrogated by ovariectomy (FIG. 15a). However, if progesterone was administered to maintain pregnancy, normal ISP2 expression was observed (FIG. 15b). A similar maintenance of ISP2 gene expression was not observed in the presence of estrogen alone (FIG. 15c). These results suggested a requirement for progesterone in maintaining ISP2 gene expression during pregnancy.

In order to confirm the requirement of progesterone for uterine ISP2 gene expression, mice were treated with RU486 on day three of pregnancy or pseudo-pregnancy and analyzed for gene ISP2 expression in uterine sections. When sacrificed on the following day, normal ISP2 mRNA staining was observed in vehicle treated control mice (FIG. 16a, c). However, in mice treated with the antiprogestin, ISP2 mRNA staining was not observed (FIG. 16b, d). These results established the necessity of progesterone for maintaining ISP2 gene expression in pregnancy.

ISP2 gene expression could be induced by progesterone after the cessation of pregnancy by ovariectomy (FIG. 15d). In the absence of progesterone maintenance after ovariectomy, ISP2 gene expression was not observed (data not shown). However, if pregnancy failure was induced by ovariectomy, ISP2 gene expression could still be induced up to 14 days following ovariectomy (FIG. 15d). These results confirmed that after ovariectomy and a long absence of progesterone signaling, the uterus remains responsive to progesterone, and suggested that ISP2 gene expression is induced by progesterone. Similar experiments also demonstrated that estrogen had neither a stimulatory or inhibitory effect on ISP2 gene expression. Following ovariectomy in the absence of progesterone maintenance, it was observed that estrogen did not have a stimulatory effect on ISP2 gene expression (FIG. 15f). Moreover, administration of estrogen in combination with progesterone after induced pregnancy failure resulted in no significant alteration of ISP2 gene expression (FIG. 15d) over that of progesterone treatment alone. These results suggested that progesterone alone is necessary and sufficient to bring about maximal ISP2 gene expression.

### Example 11

### ISP1 and ISP2 Immunization Inhibits Pregnancy

To investigate the effect of ISP1 and ISP2 immunization on pregnancy, fusion proteins of ISP1 and ISP2 were prepared and used to immunize mice. The mice were then mated, and the effect of the immunization was determined.

To prepare fusion proteins, regions of dissimilarity between ISP1 and ISP2 (FIG. 17) were amplified by PCR for the generation of pGEX fusion proteins. ISP1 and ISP2 amplicons bearing 5′- EcoRI and 3′-BamHI subcloning sites were generated from ISP1 and ISP2 full length cDNA clones (lOng template DNA) using the primers pairs: (ISP1-5′(BamHI): 5′-GCGGATCCCA GGACAACAAGAGC-3′; SEQ ID NO:20 and ISPI-3′(EcoRI): 5′-GCGAATTCAGCTTTGTGCTCGTC-3′; SEQ ID NO:21), and (ISP2-5′(BamHI): 5′-GCGGATCCGACAACTCACTTTGT-3′; SEQ ID NO:22 and ISP2-3′(EcoRI): 5′-GCGAATTCGTGGTACATCTCCTC-3′; SEQ ID NO:23), and 35 cycles of PCR (95° C.-30s, 55° C.-30s, 72° C.-30s) using Taq polymerase (Sigma) in 10 mmol Tris-HCl (pH 8.3), 50 mmol KCl, 1.5 mmol MgCl2, 130 mmol dNTPs. The resulting amplicons were precipitated with ethanol, cleaved flanking 5′ BamHI and 3′ EcoRI sites designed in the primer ends, eluted from a 1% (w/v) agarose gel and cloned into BamHI/EcoRI cut pGEX2T (Pharmacia). The inserts of individual clones were screened by restriction analysis and dye-terminator sequenced (Applied Biosystems) to identify clones bearing the correct ISP1 or ISP2 fusion genes.

Plasmids were derived from resulting clones by miniprep and subsequently introduced into E.coli BL-21 for induction of protein. 100 ml cultures of both fusion clones were cultured to mid log phase in 2 X YT+ampicillin (50 mg/ml) and treated with IPTG (0.5 mM) overnight at 30° C. to induce the expression of both the GST-ISP1 and GST-ISP2 fusion proteins. Aliquots of the cells were lysed by sonication and analyzed by SDS-PAGE alongside equivalent parallel lysates of the pGEX-2T plasmid alone (FIG. 17). For purification of the fusion proteins, inclusion bodies were isolated by centrifugation and separated by preparative SDS-PAGE. Fusion protein bands were cut out of gel following Coomassie blue staining, were electroeluted into a dialysis membrane, dialyzed against PBS, lyophilized, then reconstructed in 2 ml PBS. Protein concentrations were estimated by SDS PAGE using marker standards.

10 mg of ISP1 and ISP2 GST fusion protein was mixed in Freunds complete adjuvant (100 ml) for initial immunization of mice. BALB C female mice (Charles River), six weeks old (15 in total) were immunized by intraperitoneal injection of both ISP1 and ISP2 GST fusion proteins (10 mg) in Freunds complete adjuvant (100 ml per injection). In parallel, five females were mock immunized using Freunds complete adjuvant alone as negative controls. Mice were boosted four times at three week intervals by intraperitoneal injection using both fusion proteins (10 mg) in Freunds incomplete adjuvant (100 ml per injection). Prior to the third boost, 100 μl of blood was collected from the tail vein of each mouse and used in western blots against ISP1 and ISP2 fusion protein to confirm that an immune response had specifically occurred in each experimental mouse.

One week following the final boost, BALB C male mice were mated with immunized or mock immunized female mice to investigate the effect of ISP immunization on female fertility. Following the identification of vaginal plugs, mice were sacrificed by cervical dislocation at mid to late gestation to confirm the presence or absence of embryos. The results (Table 2) indicate that ISP1 and ISP2 immunization significantly reduced the number of embryos in the treated animals (Mice A1-A5, B1-B5 and C1-C5), while control mice (D1-D5) which received only Freunds adjuvant had normal numbers of embryos. Therefore, ISP1 and ISP2 immunization reduced the ability of mice to become pregnant, confirming that ISP1 and ISP2 are essential for fertility.

TABLE 2
The Effect of ISP1/ISP2 on Pregnancy

| Mouse | Number of Embryo |
|---|---|
| A1 | 0 |
| A2 | 7 |
| A3 | 9 |
| A4 | 7 |
| A5 | 0 |
| B1 | 0 |
| B2 | 0 |
| B3 | 0 |
| B4 | 5 |
| B5 | 0 |
| C1 | 0 |
| C2 | 5 |
| C3 | 4 |
| C4 | 4 |
| C5 | 0 |
| D1 | 11 |
| D2 | 9 |
| D3 | 9 |
| D4 | 10 |
| D5 | 9 |

The mice in groups A, B and C were immunized with ISP1- and ISP2-GST fusion proteins in Freunds adjuvant. The control mice (Group D) were treated in parallel with Freunds adjuvant only.

### Example 12

### Generation of Mouse ISP1 and ISP2 Genomic Sequences

To isolate the genomic sequences for ISP1 and ISP2, a Sau3A partial mouse ES cell genomic library in the vector lambda TK (Woltjen et al., 2000) was screened by plaque hybridization using the mouse ³²P-labeled ISP1 and ISP2 cDNA clones as probes. Standard hybridization conditions (5×SSC, 5×Denharts, 0.5%SDS, 65° C.) and stringent washing conditions (0.1×SSC, 0.5% SDS, 65° C.) were used to isolate specific clones. Individual phage clones were grown in large scale using CsCl equilibrium gradient centrifugation to generate high quality DNA for DNA sequencing

The genomic regions comprising each ISP genes were amplified from the phages by PCR using primers directed between the 5′ and 3′ untranslated sequences. For ISP1, a 2.2 kb genomic fragment was isolated using the following primers and PCR conditions using taq polymerase:

5′-UTR: 5′-ATATGAATTCGACTGTTGCTCCTGGCTCTC-3′ (SEQ ID NO:28);

3′-UTR: 5′-ATATCTCGAGTGAGAAGATTGATGGCAGAT-3′ (SEQ ID NO:29); and 95° C.-3 min; [95° C.-1 min; 58° C.-1 min; 72° C.-3 min] ×35 cycles; 72° C.-7 min; 4° C. overnight.

For ISP2, a 3.8 kb genomic fragment was isolated using the following primers and PCR conditions using taq polymerase:

5′-UTR: 5′-ATATGAATTCCGTCCTGTGAGTGGTTCTCA-3′ (SEQ ID NO:30);

3′-UTR: 5′-ATATAAGCTTAGGAAGCCAGGAAACTGAGC-3′ (SEQ ID NO:31); and 95° C.-3 min; [95° C.-1 min; 63° C.-1 min; 72° C.-5 min] ×35 cycles; 72° C.-7 min; 4° C. overnight.

The PCR primers used incorporated restriction at the end of the fragments to allow subcloning into the vector pBS KS⁺ (Stratagene Inc, La Jolla, Calif.). The ISP1 genomic fragment was subcloned using 5′EcoRI and 3′XhoI. The ISP2 genomic fragment was subcloned using EcoRi and HindIII. Four representative clones for each gene were isolated via dye primer sequencing (Perkin Elmer-Applied Biosystems, Foster City, Calif.). Sequences for the outside ends were first collected using vector specific sequencing primers. Internal sequences were generated by sequence walking using primers specific to the exons of either ISP1 or ISP2. Additionally, the 5′ and 3′ most ends of each gene were sequenced directly from the initial bacteriophage genomic clones.

The genomic sequences are shown in FIG. 18 (ISP1 genomic; SEQ ID NO:25) and FIG. 19 (ISP2 genomic; SEQ ID NO:26), respectively.

### Example 13

### Identification of Human ISP2 cDNA Sequences

The human orthologue of ISP2 was predicted from human genomic sequences generated by high throughput genomic DNA sequencing. Mouse ISP2 cDNA sequence was used in blastn and blastx searches (Altschul et al., 1997) against the NCBI database (www.ncbi.nlm.nih.gov/blast/) to identify a putative human orthologue. Both searches led to a strong match: AE006466 Homo sapiens 16p13.3 sequence section 5 of 8 (Daniels et al. 2001), in the region lying between nucleotides 197,185 and 199,032. Human ISP2 cDNA sequences within this region was predicted using GenScan (Burge and Karlin, 1997) and aligned with ISP2 proteins sequences using the Blastp alignment tool (Altschul et al., 1997). Expression of this orthologue was confirmed by RT-PCR using human uterine and placental RNAs and primers directed against the predicted human ISP2 gene:

5′-CTGGGTGCGGATGTGGCCCTGCTCC-3′ (SEQ ID NO:32);

5′-CTGCAGGCGGTAGGGCGGCGGCAGCG-3′ (SEQ ID NO:33); and

(95° C.-1 min; 58° C.-1 min; 72° C.-3 min)×35 cycles; 72° C.-7 min; 4° C. overnight.

The amino acid sequence of the human ISP2 (SEQ ID NO:27) thus identified is shown in FIG. 20, along with a comparison of the human ISP2 (hISP2; SEQ ID NO:27), ISP1 and ISP2. The cDNA sequence of human ISP2 (SEQ ID NO:34) is shown in FIG. 21.

We claim:

1. An isolated DNA encoding an Implantation Serine Proteinase (ISP) protein.

2. The DNA of claim 1, wherein the DNA is selected from the group consisting of
(a) a DNA having at least about 60% sequence identity with SEQ ID NO:1 or SEQ ID NO:2; and
(b) a DNA capable of hybridizing with SEQ ID NO:1 or SEQ ID NO:2 under a stringency equivalent to 0.5×SSC and 50° C.

3. The DNA of claim 1, wherein the DNA is selected from the group consisting of
(a) a DNA having at least about 70% sequence identity with SEQ ID NO:1 or SEQ ID NO:2; and
(b) a DNA capable of hybridizing with SEQ ID NO:1 or SEQ ID NO:2 under a stringency equivalent to 0.5×SSC and 55° C.

4. The DNA of claim 1, wherein the DNA is selected from the group consisting of
(a) a DNA having at least about 80% sequence identity with SEQ ID NO:1 or SEQ ID NO:2; and
(b) a DNA capable of hybridizing with SEQ ID NO:1 or SEQ ID NO:2 under a stringency equivalent to 0.1×SSC and 55° C.

5. The DNA of claim 1 comprising a sequence selected from the group consisting of SEQ ID NO:1 and SEQ ID NO:2.

6. A vector comprising the DNA of claim 1.

7. A cell comprising the vector of claim 6.

8. The cell of claim 7 wherein the cell is a eukaryotic cell.

9. A purified ISP protein.

10. The protein of claim 9 wherein the protein is a recombinant protein.

11. The protein of claim 9 having at least about 50% sequence identity with SEQ ID NO:3 or SEQ ID NO:4.

12. The protein of claim 9 having at least about 60% sequence identity with SEQ ID NO:3 or SEQ ID NO:4.

13. The protein of claim 9 having at least about 70% sequence identity with SEQ ID NO:3 or SEQ ID NO:4.

14. The protein of claim 9 having at least about 80% sequence identity with SEQ ID NO:3 or SEQ ID NO:4.

15. The protein of claim 9 having at least about 90% sequence identity with SEQ ID NO:3 or SEQ ID NO:4.

16. The protein of claim 9 comprising a sequence selected from the group consisting of SEQ ID NO:3 and SEQ ID NO:4.

17. A method for producing a recombinant ISP protein, comprising constructing an expression vector comprising a DNA encoding an ISP protein, introducing the expression vector into a suitable cell and selecting transformants, culturing the transformants under conditions that result in production of the ISP protein, and recovering the ISP protein.

18. The method of claim 16, wherein the DNA is selected from the group consisting of
(a) a DNA having at least about 60% sequence identity with SEQ ID NO:1 or SEQ ID NO:2; and
(b) a DNA capable of hybridizing with SEQ ID NO:1 or SEQ ID NO:2 under a stringency equivalent to 0.5x SSC and 50° C.

19. The method of claim 16, wherein the DNA is selected from the group consisting of
(a) a DNA having at least about 70% sequence identity with SEQ ID NO:1 or SEQ ID NO:2; and
(b) a DNA capable of hybridizing with SEQ ID NO:1 or SEQ ID NO:2 under a stringency equivalent to 0.5×SSC and 55° C.

20. The method of claim 16, wherein the DNA is selected from the group consisting of
(a) a DNA having at least about 80% sequence identity with SEQ ID NO:1 or SEQ ID NO:2; and
(b) a DNA capable of hybridizing with SEQ ID NO:1 or SEQ ID NO:2 under a stringency equivalent to 0.0×SSC and 55° C.

21. A method for contraception in a mammal, comprising immunizing the mammal with an ISP protein, a nucleic acid encoding an ISP protein, or a fragment of the ISP protein or nucleic acid.

22. The method of claim 21 wherein the protein is at least about 70% sequence identity with SEQ ID NO:3 or SEQ ID NO:4.

23. The method of claim 21 wherein the protein is at least about 80% sequence identity with SEQ ID NO:3 or SEQ ID NO:4.

24. The method of claim 21 wherein the protein is at least about 90% sequence identity with SEQ ID NO:3 or SEQ ID NO:4.

25. The method of claim 21 wherein the protein comprises SEQ ID NO:3 or SEQ ID NO:4.

26. The method of claim 21 wherein the ISP protein is a fusion protein.

27. The method of claim 21 wherein an adjuvant is also administered into the mammal.

28. An isolated antibody recognizing an epitope of ISP1 or ISP2.

29. The antibody of claim 28 wherein the antibody is monoclonal.

30. The antibody of claim 28 wherein the antibody is polyclonal.

31. The antibody of claim 28 capable of inhibiting an ISP protein.

32. A pharmaceutical composition comprising an ISP protein or a nucleic acid encoding an ISP protein.

33. The pharmaceutical composition of claim 32 further comprising an adjuvant.

34. The pharmaceutical composition of claim 32 wherein the ISP protein is at least about 70% sequen ce identity with SEQ ID NO:3 or SEQ ID NO:4.

35. The pharmaceutical composition of claim 32 wherein the ISP protein is at least about 80% sequence identity with SEQ ID NO:3 or SEQ ID NO:4.

36. The pharmaceutical composition of claim 32 wherein the ISP protein is at least about 90% sequence identity with SEQ ID NO:3 or SEQ ID NO:4.

37. The pharmaceutical composition of claim 32 wherein the ISP protein comprises SEQ ID NO:3 or SEQ ID NO:4.

38. A method for contraception in a mammal, comprising administering into the mammal an effective amount of an inhibitor of ISP1 or ISP2 under conditions that result in contraception.

39. The method of claim 38 wherein the inhibitor is an antibody.

40. The method of claim 38 wherein the inhibitor is an antisense oligonucleotide.

41. A pharmaceutical composition useful for contraception, comprising an inhibitor of ISP1 or ISP2.

42. The pharmaceutical composition of claim 41 wherein the inhibitor is an antibody.

43. The pharmaceutical composition of claim 41 wherein the inhibitor is an antisense oligonucleotide.

44. A method for screening for inhibitors of ISP1 or ISP2, comprising providing an assay for ISP1 or ISP2 activity, determining the effect of a candidate compound on ISP1 or ISP2 activity in the assay, and identifying an inhibitor as a candidate compound capable of inhibiting ISP1 or ISP2 activity.

45. The method of claim 44 wherein the identified inhibitor is useful in contraception.

46. A method for diagnosing infertility of a mammal, comprising providing an assay for ISP1 or ISP2, providing a biological sample from the mammal, subjecting the biological sample to the assay, and diagnosing the mammal as having infertility if ISP1 or ISP2 activity/level is low.

47. A method for treating or ameliorating infertility, comprising providing an effective amount of an ISP protein or a nucleic acid encoding an ISP protein to a mammal.

48. The method of claim 47 wherein the ISP protein has at least about 60% sequence identity with SEQ ID NO:3 or SEQ ID NO:4.

49. The method of claim 47 wherein the ISP protein has at least about 70% sequence identity with SEQ ID NO:3 or SEQ ID NO:4.

50. The method of claim 47 wherein the ISP protein has at least about 80% sequence identity with SEQ ID NO:3 or SEQ ID NO:4.

51. The method of claim 47 wherein the ISP protein has at least about 90% sequence identity with SEQ ID NO:3 or SEQ ID NO:4.

52. The method of claim 47 wherein the ISP protein comprises SEQ ID NO:3 or SEQ ID NO:4.

53. A method for enhancing implantation of a cultured embryo comprising contacting the cultured embryo with an ISP protein prior to placement of the cultured embryo in the uterus of a female mammal.

54. The method of claim 53 wherein the mammal is human.

55. The method of claim 53 wherein the ISP protein is selected from the group consisting of ISP1 and ISP2.
